(19) United States
(12) Patent Application Publication
(10) Pub. No.: US 2025/0000672 A1
(43) Pub. Date: Jan. 2, 2025

(54) METHODS FOR REPAIRING CARTILAGE DEFECTS

(71) Applicant: Vericel Corporation, Cambridge, MA (US)

(72) Inventors: Graham Smith, Newburyport, MA (US); Felix Ernesto Robles Toth, Duxbury, MA (US); Richard P. Rodgers, Hudson, MA (US); Robert Bruce Mackinnon, III, Carver, MA (US)

(21) Appl. No.: 18/293,884

(22) PCT Filed: Aug. 12, 2022
(86) PCT No.: PCT/US22/40207

§ 371 Date: Jan. 31, 2024

Related U.S. Application Data
(60) Provisional application No. 63/235,473, filed on Aug. 20, 2021.

(51) Int. Cl. A61F 2/46; A61F 2/30; A61L 27/38; A61L 27/44; A61L 27/58

(52) U.S. Cl. CPC A61F 2/4618; A61L 27/3817; A61L 27/3852; A61L 27/44; A61L 27/58; A61F 2002/30766; A61F 2002/4635; A61L 2430/06

(57) ABSTRACT

The present disclosure describes methods for repairing cartilage defects by arthroscopic surgery to deliver cultured chondrocyte implants to defect sites. The implants may include bioresorbable matrices seeded with chondrocytes that are shaped to match cartilage defect sites, and which may be delivered to the cartilage defect sites by methods described in the present embodiments.

### CROSS-REFERENCE TO RELATED APPLICATIONS

This application claims priority to U.S. Provisional Application No. 63/235,473, filed Aug. 20, 2021, entitled “Methods for Repairing Cartilage Defects”, the contents of which are incorporated herein by reference in their entirety.

### BACKGROUND

Chondral and osteochondral lesions such as, for example, focal lesions in the load bearing region of a knee's articular cartilage greatly increase the risk for osteoarthritis. This type of lesion occurs frequently from, for example, trauma, participation in sports, osteochondritis dissecans, etc. The capacity for spontaneous repair of chondral lesions is minimal, due in part to the limited blood supply to cartilage tissue. Treatment of damaged cartilage requires replacement of defective cartilage with healthy cartilage: autologous chondrocyte implantation strategies have been described to accomplish such replacement (Brittberg et al. Clin. Orthopaed. Red. Res. (1999) 367S: S147-S155). In such procedures, chondrocytes are harvested from a patient, expanded in cell culture to increase the number of chondrocytes, and then implanted back into the injury site of the patient.

More recent work has improved autologous implantation by seeding expanded autologous cells on a matrix in a process known as matrix-induced autologous chondrocyte implantation (MACI) (Basad et al. In: Hendrich et al., Cartilage Surgery and Future Perspectives, Thieme Verlag, 49-56 (2003)). The MACI process has been further improved to allow for the implantation of allogeneic cells, reducing the total number of necessary procedures undergone by a patient. However, the MACI procedure has typically been performed via mini-arthrotomy, an open surgical technique that generally presents a greater risk of infection, longer recovery times, and increased pain for patients when compared to less invasive surgical methods. Moreover, many patients present with defects that are amenable to treatment via minimally invasive procedures.

### SUMMARY

The present disclosure provides improved matrix-induced autologous chondrocyte implantation (MACI) technologies. For example, among other things, the present disclosure provides technologies for the arthroscopic delivery of MACI implants: in some embodiments, provided technologies are characterized in that they achieve delivery characterized by levels of cell viability comparable to those observed with non-arthroscopic delivery. Advantages of the provided methods include, for example, arthroscopic delivery that is far less invasive than open surgical strategies that have typically been used to administer MACI implants. Provided technologies, thus, represent and embody further improvements with respect to MACI technologies for the treatment of tissue defects (for example, cartilage defects, among other types of defects).

In one aspect, the present disclosures are directed to an arthroscopic surgical method including: making at least two incisions in a subject adjacent to a defect to be treated arthroscopically, the at least two incisions including a first incision and a second incision; inserting a first cannula into the first incision: preparing a defect site at a joint in the subject; shaping a template material to match the shape of the prepared defect site; shaping a cell-seeded matrix to match the shape of the shaped template material by placing the cell-seeded matrix on top of the template material with the cells facing up and cutting the cell-seeded matrix to match the shape of the template material: delivering the cell-seeded matrix to the prepared defect site through the first cannula; and fixating the delivered cell-seeded matrix using a glue.

In some embodiments, the defect includes a cartilage defect. In some embodiments, preparing the defect site includes: flushing the defect site: assessing and/or measuring the defect site: outlining the defect site: sculpting the defect site to remove damaged tissue; and debriding the cartilage down to subchondral bone.

In some embodiments, the template material includes at least one member of the group consisting of sterile aluminum foil, sterile paper, and an Esmarch bandage. In some embodiments, shaping the template material includes: (a) passing the template material through the first cannula inserted at the first incision: (b) observing the template material adjacent to the defect site: (c) removing the template material from the first cannula and cutting it to approximate the size and/or shape of the defect site based on the observations; and (d) repeating steps (a)-(c) until the template material matches the size and/or shape of the defect site.

In some embodiments, the cell-seeded matrix includes at least one of a bioresorbable material and collagen to form a matrix, and the cells are seeded on a surface of the matrix at a concentration of at least 250,000 cells/cm². In some embodiments, the cells include chondrocytes. In some embodiments, the cells are at least one of cells autologous to the subject and allogeneic cells. In some embodiments, the template material remains with the shaped cell-seeded matrix during delivery of the cell-seeded matrix to provide structural support.

In some embodiments, the glue may include a biocompatible glue and/or a fibrin glue. In some embodiments, the cell-seeded matrix includes cells seeded on one surface and no cells on another surface.

In some embodiments, delivering the cell-seeded matrix includes: grasping the cell-seeded matrix using a surgical grasper: bringing the unseeded surface of the cell-seeded matrix into contact with a proximal opening of the first cannula inserted into the first incision: pushing the cell-seeded matrix into a lumen of the cannula; and using the surgical grasper to push the cell-seeded matrix through to a distal end of the cannula.

In some embodiments, delivering the cell-seeded matrix includes: folding the cell-seeded matrix in half: grasping the folded cell-seeded matrix using a surgical grasper; bringing the folded cell-seeded matrix into contact with a proximal opening of the first cannula inserted at the first incision: pushing the folded cell-seeded matrix into a lumen of the first cannula inserted at the first incision; and using the surgical grasper to push the folded cell-seeded matrix through to the distal end of the cannula.

In some embodiments, delivering the cell-seeded matrix includes: prior to inserting the first cannula into the first incision, disposing a surgical tool in a lumen of the first cannula such that jaws of the surgical tool protrude from a distal end of the first cannula and a handle of the surgical tool protrude from a proximal opening of the first cannula; grasping the cell-seeded matrix using the jaws of the surgical tool: pulling the cell-seeded matrix into the distal end of the first cannula using the surgical tool such that the cell-seeded surface faces a lumen of the first cannula and the cell-seeded matrix is partially folded; inserting the first cannula into the first incision while the cell-seeded matrix and surgical tool are disposed within the lumen of the first cannula; and using the surgical tool to push the cell-seeded matrix out of the distal end of the first cannula such that the cell-seeded matrix unfolds.

In some embodiments, inserting the first cannula includes inserting the first cannula into a third cannula that has previously been inserted into the first incision.

In some embodiments, each cannula includes an inner diameter in a range from about 5 mm to about 20 mm and a length in a range from about 20 mm to about 240 mm.

In some embodiments, the area of the defect and the area of the cell-seeded matrix after shaping are between about 1 cm² and about 10 cm².

In another aspect, the present disclosures are directed to a surgical kit including: two or more cannulas: a cell-seeded matrix comprising a bioresorbable support matrix and a plurality of cells seeded on a surface of the bioresorbable support matrix at a concentration of at least 250,000 cells/cm²: a surgical grasper: a templating material; one or more tools for shaping the template material and the cell-seeded matrix; one or more tools for outlining, cutting, and debriding cartilage.

In some embodiments, the two or more cannulas each include an inner diameter in a range from about 5 mm to about 20 mm and a length in a range from about 20 mm to about 240 mm.

In some embodiments, the surgical grasper includes a shaft and jaws, wherein the jaws include atraumatic jaws. In some embodiments, the one or more tools for shaping include members selected from the group consisting of scissors, razor blades, scalpels, custom cutters, cutting blocks, surgical mallets, ring curettes, tweezers, and cutting needles. In some embodiments, the one or more tools for shaping include custom cutters with blades shaped as a circle or an oval.

In another aspect, the present disclosures are directed to a custom surgical device including: a handle: an adjustable knob rotatably coupled to a proximal end of the handle: a shaft coupled to the distal end of the handle: a movable joint coupled to the distal end of the shaft; and an adjustable distal end coupled to the moveable joint, such that rotating the adjustable knob causes the adjustable distal end to rotate about the moveable joint such that an angle between the adjustable distal end and the shaft changes.

In some embodiments, the adjustable distal end includes an adjustable cutter.

In some embodiments, the custom surgical device further includes at least one cable mechanically coupling the adjustable distal end to the adjustable knob, such that the at least one cable is disposed within and/or through each of the moveable joint, the shaft, and the handle.

In some embodiments, the shaft is coupled to the handle at a fixed angle.

In some embodiments, the defect includes a cartilage defect: preparing the defect site includes removing damaged tissue and debriding the cartilage down to subchondral bone: the cell-seeded matrix includes a collagen support matrix and chondrocyte cells seeded on a side of the collagen support matrix at a concentration of at least 250,000 cells/cm²: the template material includes one of a sterile aluminum foil, a sterile paper, or a sterile bandage; shaping the template material includes cutting the template material to match the shape and size of the defect site; shaping the cell-seeded matrix includes cutting the cell-seeded matrix to match the shape and size of the template material; and the glue includes a biocompatible fibrin glue.

In some embodiments, the method further includes: inserting a second cannula into the second incision; and disposing an arthroscope within the second cannula.

### BRIEF DESCRIPTION OF THE DRAWINGS

The present teachings described herein will be more fully understood from the following description of various illustrative embodiments, when read together with the accompanying drawing. It should be understood that the drawings described below are for illustration purposes only and are not intended to limit the scope of the present teachings in any way.

FIG. 1A, FIG. 1B, FIG. 1C, and FIG. 1D depict a series of photographs illustrating the surgical steps of arthroscopic delivery of a cell-seeded matrix to a tissue defect in a human knee joint, according to aspects of the present embodiments.

FIG. 2A, FIG. 2B, FIG. 2C, and FIG. 2D depict a series of photographs illustrating the “unfolded” method of use of a cannula to arthroscopically deliver a composition to a surgical site, according to aspects of the present embodiments.

FIG. 3A, FIG. 3B, FIG. 3C, and FIG. 3D depict a series of photographs illustrating the “folded” method of use of a cannula to arthroscopically deliver a composition to a surgical site, according to aspects of the present embodiments.

FIG. 4A, FIG. 4B, FIG. 4C, FIG. 4D, and FIG. 4E depict a series of photographs illustrating the “distal loading” method of use of a cannula to arthroscopically deliver a composition to a surgical site, according to aspects of the present embodiments.

FIG. 5A, FIG. 5B, and FIG. 5C depict an exemplary series of photographs of cell-seeded matrices stained with 3-(4,5-dimethylthiazol-2-yl)-2,5-diphenyltetrazolium bromide (MTT) to assess metabolic activity of cells following surgical grasping and delivery to a surgical site by several delivery different methods, according to aspects of the present embodiments.

FIG. 6A and FIG. 6B depict cell viability data obtained from matrix-seeded cell samples following simulated delivery of the matrix-seeded cells to a site in the knee of a human cadaver through a variety of different methods, according to aspects of the present embodiments.

FIG. 7A, FIG. 7B, FIG. 7C, and FIG. 7D depict multiple views of an adjustable cutting device for use according to aspects of the present embodiments.

FIG. 8A, FIG. 8B, and FIG. 8C depict multiple views of an adjustable cutting device for use according to aspects of the present embodiments, especially relating to adjusting an angle of a cutting component.

FIG. 9A, FIG. 9B, and FIG. 9C depict multiple views of an adjustable cutting device for use according to aspects of the present embodiments, especially relating to adjusting a size of a cutting component.

FIG. 10A, FIG. 10B, FIG. 10C, and FIG. 10D depict multiple views of a custom cutting device for use according to aspects of the present embodiments, especially relating to procedures or processes to shape, score, mark, and/or cut tissue into a desired shape.

FIG. 11A, FIG. 11B, and FIG. 11C depict close-up views of the distal end of the custom cutting device depicted in FIG. 10A, FIG. 10B, FIG. 10C, and FIG. 10D, for use according to aspects of the present embodiments. In particular, various configurations of the cutting teeth during operation of the device are illustrated.

FIG. 12 depicts a flowchart of a method of obtaining autologous chondrocytes and preparing implants, according to aspects of the present embodiments.

FIG. 13 depicts a flowchart of an arthroscopic surgical method to implant a cell-seeded matrix implant at a defect site, according to aspects of the present embodiments.

FIG. 14 depicts a flowchart of a method to deliver a cell-seeded matrix by an unfolded method, according to aspects of the present embodiments.

FIG. 15 depicts a flowchart of a method to deliver a cell-seeded matrix by a folded method, according to aspects of the present embodiments.

FIG. 16 depicts a flowchart of a method to deliver a cell-seeded matrix by a distal loading method, according to aspects of the present embodiments.

### DEFINITIONS

As used herein, the term “about,” as used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the term “adult” refers to a human eighteen years of age or older. In some embodiments, a human adult has a weight within the range of about 90 pounds to about 250 pounds.

As used herein, the term, “associated with” refers to two events or entities when presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to a disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another: in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “biocompatible” refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro does not result in substantial cell death, and/or their administration in vivo does not induce significant inflammation or other such adverse effects.

As used herein, the term “chondrocytes” or “cartilage cells,” refers to cells that are capable of expressing biochemical markers characteristic of chondrocytes, including but not limited to type II collagen, aggrecan, chondroitin sulfate and/or keratin sulfate. In some embodiments, chondrocytes, or cartilage cells, express morphologic markers characteristic of smooth muscle cells, including but not limited to a rounded morphology in vitro. In some embodiments, chondrocytes, or cartilage cells, are able to secrete type II collagen in vitro. In some embodiments, chondrocytes, or cartilage cells, are able to secrete aggrecan in vitro. In some embodiments, chondrocytes, or cartilage calls, are able to generate tissue or matrices with hemodynamic properties of cartilage in vitro.

As used herein the term “ex vivo” refers to events that occur in tissue outside of or removed from a multi-cellular organism, such as a human and a non-human animal. In the context of cell-based systems, the term may be used to refer to events that occur within an isolated tissue sample taken from an organism (as opposed to, for example, in vivo systems).

As used herein, the term “extracellular” refers to a molecule, substance, or process that is situated or taking place outside of a cell or group of cells. In the context of cell-based systems, the term may be used to refer to natural biological matter found adjacent to and outside of a cell or group of cells (e.g., “extracellular matrix”).

As used herein, the term “defect” refers to an abnormality or imperfection, for example, in tissue in a joint of a subject. In some embodiments, a defect is a cartilaginous defect. In some embodiments, a defect is a defect in tissue in an articulating joint, for example, a knee joint. In some embodiments, a defect is a chondral defect. In some embodiments, a defect is an osteochondral defect. In some embodiments, a defect may have a size ranging from about 0.1 to about 10 cm². In some embodiments, a defect may have a size that is greater than 10 cm².

As used herein, the term “density” refers to an average number of a substance, for example, cells or another object, per unit area of volume. In some embodiments, density is cell density, i.e., number of cells per unit of surface area. In some embodiments, an average density is approximated by dividing a number of cells seeded by a macroscopic surface area of a surface on which they are grown. In some embodiments, a surface is two-dimensional. In some embodiments, a surface is three-dimensional.

As used herein the term “in vitro” refers to events that occur in an artificial environment, e.g., in a test tube or reaction vessel, in cell culture, etc., rather than within a multi-cellular organism.

As used herein the term “in vivo” refers to events that occur within a multi-cellular organism, such as a human and a non-human animal. In the context of cell-based systems, the term may be used to refer to events that occur within a living cell (as opposed to, for example, in vitro systems).

As used herein, the term “medium” refers to components which support growth or maintenance of cells in culture. In some embodiments, this may include traditional liquid cell culture medium and an additional factor. In some embodiments, additional factors may include, for example, serum, antibiotics, growth factors, pharmacological agents, buffers, pH indicators and the like. In some embodiments, a medium may be used in a process to isolate cells (e.g., chondrocytes and/or chondrocyte precursors) from a tissue sample (e.g., a cartilage sample). In some embodiments, tissue is mechanically disrupted (e.g., chopped, minced, blended) then combined with a medium. In some embodiments, a medium comprises enzymes (e.g., collagenase, protease) to digest tissue and release cells.

As used herein, the term “conditioned medium” refers to a medium which has been contacted with cells to allow for the composition of medium to be modified, for example by uptake or release of one or more metabolites, nutrients, or factors.

As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the patient is receiving or has received certain therapy to diagnose and/or to treat a disease, disorder, or condition.

As used herein, the term “seeding” refers to a process or step whereby cells are brought into contact with a support matrix, and adhere (with or without an adhesive) to a support matrix (e.g., a collagen membrane) for a period of time. Seeded cells may divide and/or differentiate on a support matrix. In some embodiments, cells are seeded onto a support matrix prior to being implanted into a subject.

As used herein, the term “subject” refers to an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered. In some embodiments, a subject is a donor of a biological sample, tissue and/or material.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “substantially free of endotoxin” refers to a level of endotoxin per dose of a composition that is less than is allowed by the FDA for a biologic product (i.e., total endotoxin of 5 EU/kg body weight per hour, which for an average 70 kg person is 350 EU per total dose).

As used herein, the term “substantially free of mycoplasma and/or microbial contamination” refers to a negative reading for a generally accepted test of contamination known to those skilled in the art. For example, mycoplasma contamination is determined by subculturing a product sample in broth medium and distributing the culture over agar plates on days 1, 3, 7, and 14 at 37° C. with appropriate positive and negative controls. In some embodiments, mycoplasma contamination is determined using a real-time PCR method. The product sample appearance is compared microscopically at 100×, to that of a positive and negative control. Additionally, presence of mycoplasma contamination may be detected by inoculation of an indicator cell culture, which is incubated for 3 and 5 days then examined at 600× by epifluorescence microscopy using a DNA-binding fluorochrome. The composition is considered satisfactory if agar and/or broth media procedure and indicator cell culture procedure show no evidence of mycoplasma contamination. In some embodiments, an assay that may be utilized to assess a level of microbial contamination may be or include the U.S. Pharmacopeia (USP) Direct Transfer Method. This involves inoculating a sample into a tube containing tryptic soy broth media and fluid thioglycollate media. Tubes are observed periodically for a cloudy appearance (turbidity) during a specified period (e.g., 14 days) of incubation. A cloudy appearance on any day in either medium indicates contamination, with a clear appearance (no growth) indicating that a composition may be considered to be substantially free of contamination. In some embodiments, an approved alternative to a USP method for detection of microbial contamination is used, for example, a BacT/ALERT test using different media formulations.

As used herein, the term “surface area” refers to, for example, square area, cm², or to the macroscopic surface area of a substrate.

As used herein, the term “treatment” (also “treat” or “treating”) refers to administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DETAILED DESCRIPTION OF CERTAIN EMBODIMENTS

Injuries to joints occur frequently from physical activity, for example, including but not limited to repetitive and excessive motions, overstretching, and physical trauma. Treatments for joint injuries often include surgery. Tissue, including cartilage, in the interior of an articulating joint is often difficult to access surgically, presenting challenges to treating patients with damage to joint cartilage. Certain current therapeutic intervention strategies typically involve removing damaged or dislodged cartilage from the joint. Such treatments typically provide temporary relief from symptoms of the injury, but they do not treat the origin of the lesion or defect, and, in particular, do not prevent progressive degradation of the cartilage.

The present disclosure provides improved technologies useful for treating tissue defects in articulating joints. In particular, the present disclosure provides improved matrix-induced autologous chondrocyte implantation (MACI) technologies useful for repairing a tissue defect in an articulating joint in a human subject. For example, among other things, the present disclosure provides technologies for the arthroscopic delivery of MACI implants. In some embodiments, provided technologies are characterized in that they achieve delivery characterized by cell number and viability comparable to those observed with non-arthroscopic delivery. Advantages of the provided methods include, for example, arthroscopic delivery that is far less invasive than open surgical strategies, which have typically been used to administer MACI implants. Provided technologies, thus, represent and embody further improvements with respect to MACI technologies for the treatment of cartilage defects.

### Matrix-Induced Autologous Chondrocyte Implantation (MACI)

Matrix-induced autologous chondrocyte implantation (MACI) is a surgical procedure used to treat symptomatic, full-thickness chondral lesions of articulating joints. MACIR also refers to a commercial product owned by Vericel Corporation, known as autologous cultured chondrocytes on porcine collagen membrane. MACI is a registered trademark of Vericel Corporation, but is also used herein to describe a process, and thus is not always denoted with the registration symbol. The MACI procedure is performed most commonly on the knee. MACI improves on the limitations of previous methods to treat chondral defects using implanted chondrocytes, including the risk of uneven chondrocyte distribution at the time of implantation and graft hypertrophy. Given the compliant properties of the scaffold or matrix on which chondrocytes are seeded before delivery to a patient in need, the graft can be easily shaped to treat irregular chondral defects and applied to articular surfaces with multiplanar geometry (e.g., trochlea) (Jones & Cash, 2019, Arthroscopy Techniques, 8 (3), 259-266).

Restorative treatment options for symptomatic, full-thickness chondral and osteochondral lesions of the knee continue to evolve with advancements in our understanding of cartilage biology and surgical techniques. Since the initial description by Brittberg et al., in 1994, autologous chondrocyte implantation (ACI) has gained widespread use, and surgical utilization in the United States has nearly doubled over the past decade. Although the long-term clinical results of first-generation techniques have demonstrated sustained functional improvement, there were significant technical challenges and adverse events related to the requisite use of a periosteal patch over the defect. A large number of patients demonstrated arthrofibrosis and graft hypertrophy, which necessitated additional surgical procedures to address these complications (Jones & Cash, 2019, Arthroscopy Techniques, 8 (3), 259-266). Ultimately, the use of periosteum was largely abandoned in favor of a bioabsorbable collagen membrane cover in 2007, significantly reducing the rate of graft hypertrophy and the rates of reoperation (Jones & Cash, 2019, Arthroscopy Techniques, 8 (3), 259-266).

More recent ACI techniques, including MACI, use cell-loaded membranes to avoid graft-related complications and simplify the surgical technique. The MACIR scaffold (Vericel Corporation, Cambridge, MA) may use a porcine type I/III collagen membrane seeded with autologous chondrocytes at a density ranging between 250,000 and 1 million cells/cm². In a recent report of the Superiority of MACI Implant Versus Microfracture Treatment trial, clinical outcomes following the treatment of chondral defects (≥3 cm²) with MACIR were clinically superior at 5 years compared with microfracture treatment (Brittberg et al., 2018, Am. J. Sports Med., 46, 1343-1351). Additional case series have reported similar mid- and long-term results (Jones & Cash, 2019, Arthroscopy Techniques, 8 (3), 259-266).

### Cells

In some embodiments, the present disclosure utilizes cells from a human or non-human (xenograft) source. In some embodiments, utilized cells are human cells.

In some embodiments, utilized cells are autologous in that they are obtained from the same subject to whom cell-seeded matrix compositions are administered as described herein. In some embodiments, utilized cells are allogeneic in that they are isolated from tissue of a first subject, who is a different subject from that into whom cell-seeded matrix compositions may be administered.

In some embodiments, cells may be obtained from tissue harvested from a living source (e.g., a living human). In some embodiments, cells may be obtained from tissue harvested from adult organism (e.g., an adult human). In some embodiments, cells may be obtained from tissue harvested from a human younger than 18 years of age. Alternatively or additionally, in some embodiments, cells may be obtained from tissue harvested from a deceased source (e.g., from a cadaver). In some embodiments, cells may be obtained from tissue harvested from a living non-human organism.

In some embodiments, utilized cells comprise chondrocytes. In some embodiments, utilized cells comprise human chondrocytes.

In some embodiments, a cell preparation utilized in accordance with the present disclosure may be characterized e.g., to confirm one or more features of cell identity and/or to exclude one or more contaminants or undesirable properties, etc. For example, in some embodiments, a preparation that is or comprises chondrocytes may be assessed for expression of one or more chondrocyte markers (e.g., to determine whether expression of such marker is above a predetermined threshold and/or is comparable to that observed in an appropriate reference preparation) and/or one or more fibroblast markers (e.g., to determine whether expression of such marker is below a predetermined threshold and/or is comparable to that observed in an appropriate reference preparation). In some embodiments, a chondrocyte marker may be or comprise HAPLN1, MGP, EDIL3, WISP3, AGC1, COMP, COL2A1, COL9A1, COL11A1, LECT1, 81008, CRTAC1, SOX9, and NEBL.

Cells for use according to the technologies of the present disclosure may be obtained from a biological sample, such as, for example, a tissue, cell culture, or other material, that may or may not contain chondrocytes.

In some embodiments, a cell culture may be grown from cells released from a cartilage biopsy. For example, cartilage cells may be cultured from a cartilage biopsy of a patient receiving an implant. Carticel® autologous chondrocyte product (Vericel Corporation, Cambridge, MA) is an example of a cultured chondrocyte product. In some embodiments, a cell culture comprises a collagen matrix loaded with chondrocytes. Such chondrocytes may be obtained from a cartilage biopsy and cultured prior to being loaded on the matrix, e.g., as used in the MACIR implant product.

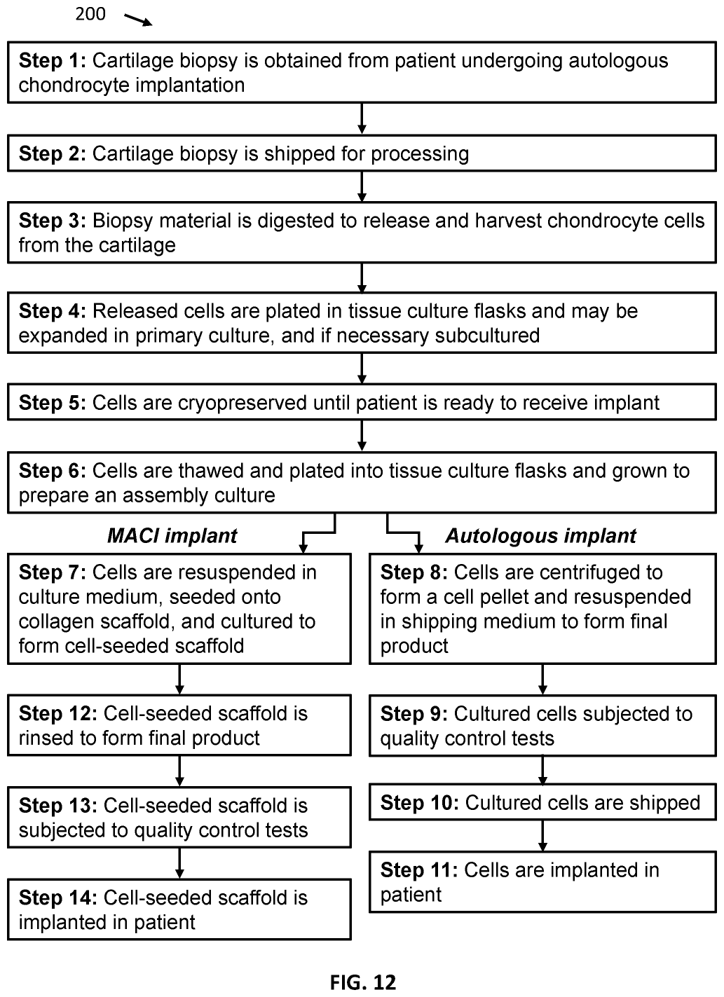

In some embodiments, autologous chondrocytes may be expanded in culture prior to implantation to the subject from which they were isolated. A method of expanding cells in culture and preparing them for use in implants is shown in FIG. 12. In step 1, a cartilage biopsy from a patient undergoing autologous chondrocyte implantation may be shipped for processing (step 2). Biopsy material is digested at step 3 to release and harvest chondrocytes from the cartilage. The released cells are plated in tissue culture flasks and may be expanded in primary culture at step 4, and if necessary, subcultured. Once the cells reach an adequate number, they can be, optionally, cryopreserved at step 5 until a patient is ready to receive an implant. Once a patient is ready to receive cells, the cells may be thawed and plated into tissue culture flasks and grown to prepare an assembly culture (step 6). For use in an autologous chondrocyte implant, if a sufficient number of cells are obtained in the assembly culture, the cells may be centrifuged to form a cell pellet and resuspended in shipping medium, which is the “final product”, such as, for example, the Carticel® autologous chondrocyte product (step 8). This “final product” may be subjected to a number of quality control tests, including for example, a sterility test, a cell viability test, an endotoxin test, a mycoplasma test, and/or a culture composition test (step 9) to ensure that the cultured cells contain a sufficient number of chondrocytes. If the cultured cells pass all tests, they may be shipped (step 10) to the patient for implantation (step 11).

Alternatively, when the assembly culture from step 6 is to be used in a MACI® implant, the cells may be resuspended in culture medium, seeded onto a collagen scaffold, and cultured for 4 days (step 7). At the end of the culture period, cells may be rinsed with shipping medium to produce a final product for MACI® implants (step 12). This product may also be subjected to quality control tests (step 13) before being implanted in a patient (step 14). Accordingly, whether the final product is a suspension of cultured chondrocytes, such as Carticel® autologous chondrocytes, or the final product is a scaffold-seeded product for MACI® implants, evaluation of cell identity may be useful as a lot identification assay or lot release assay, to confirm the composition of a cell culture as containing chondrocytes prior to shipment of the culture.

In some embodiments, RNA expression levels for genes overexpressed by chondrocytes (e.g., HAPLN1) may be measured in cultured cells. In some embodiments, RNA expression for genes overexpressed by synoviocytes (e.g., MFAP5) may be measured in cultured cells. In some embodiments, RNA expression levels may be presented as a ratio of expression of a chondrocyte marker (e.g., HAPLN1) versus expression of a synoviocyte marker (MFAP5). In some embodiments, cultured chondrocytes may demonstrate relative RNA expression levels (HAPLN1 vs. MFAP5) of about −2, about −1, about 0), about +1, about +2, about +3, about +4, about +5, about +6, about +7, about +8 about +9, about +10 or more on a log scale. In some embodiments, cultured chondrocytes may demonstrate relative RNA expression levels ranging from about −2 to about +10, about −1 to about +9, about 1 to about 10, about +3 to about +8, about +5 to about +7 or ranges therein. In some embodiments, cultured synoviocytes may demonstrate relative RNA expression levels of about less than −2 on a log scale. In some embodiments, cultured synoviocytes may demonstrate relative RNA expression levels ranging from less than −2 to −10 on a log scale.

In some embodiments, chondrocytes prepared from a source cell preparation may be present in culture at a density sufficient to seed a support matrix with at least 250,000 cells/cm². In some embodiments, chondrocytes expanded in culture may be dedifferentiated when present in a monolayer culture. In some embodiments, dedifferentiated chondrocytes may exhibit a fibroblastic phenotype. In some embodiments, dedifferentiated chondrocytes may downregulate expression of a gene encoding an extracellular matrix (ECM) protein, for example, ACAN and/or COL2A1. In some embodiments, dedifferentiated chondrocytes may produce and/or secrete a lesser amount of ECM protein, for example, collagen (e.g., type II collagen) and/or aggrecan (also known as cartilage-specific proteoglycan core protein or chondroitin sulfate proteoglycan 1). Without wishing to be bound by theory, de-differentiation may occur after removal of chondrocytes from 3-dimensional cartilage matrix and is observed during expansion of cells in monolayer culture.

In some embodiments, chondrocyte preparations utilized herein comprise a sufficient number of cells to seed a support matrix. In some embodiments, chondrocyte preparations comprise at least about 3×10⁶, 4×10⁶, 5×10⁶, 6×10⁶, 7×10⁶, 8×10⁶, 9×10⁶ or more cells following a second passage. In some embodiments, chondrocyte preparations comprise at least about 3×10⁶ cells after a second passage. In some embodiments, chondrocyte preparations disclosed herein comprise at least about 1×10⁷, 2×10⁷, 3×10⁷, 4×10⁷, 5×10⁷, 6×10⁷, 7×10⁷, 8×10⁷, 9×10⁷ or more cells at a final passage. In some embodiments, chondrocyte preparations utilized herein comprise at least 1×10⁷ cells at a final passage.

In some embodiments, chondrocyte cultures are about 50%, 60%, 70%, 80%, 90%, 95%, 98% or more confluent. In some embodiments, chondrocyte cultures are about 100% confluent. In some embodiments, chondrocyte cultures are about 50% to 90% confluent.

In some embodiments, chondrocytes are seeded on a support matrix at density of at least 250,000 cells/cm², 300,000 cells/cm², 400,000 cells/cm², 500,000 cells/cm², 600,000 cells/cm², 700,000 cells/cm², 800,000 cells/cm², 900,000 cells/cm², 1,000,000 cells/cm², or more.

Among other things, the present disclosure utilizes cell preparations in which a significant percentage of cells are viable; such high viability cell preparations can materially improve, and may be required for, successful treatment of a particular lesion or defect. In some embodiments, at least 70%, 75%, 80%, 85%, 90%, 95%, 98% or more of cells present in a preparation are viable. In some embodiments, at least 90% of chondrocytes in a preparation are viable.

In some embodiments, a composition of the disclosure utilized herein may be substantially free of components used during preparation of a source cell preparation and during expansion of chondrocytes (e.g., fetal bovine serum albumin, fetal bovine serum and/or horse serum). For example, in some embodiments, a composition utilized herein comprises less than 10 μg/ml, 5 μg/ml, 4 μg/ml, 3 μg/ml, 2 μg/ml, 1 μg/ml, 0.05 μg/ml fetal bovine serum albumin. In some embodiments, a cell preparation may be substantially free of mycoplasma, endotoxin, and/or microbial (e.g., aerobic microbe(s), anaerobic microbes(s) and/or fungi) contamination. In some embodiments, a cell preparation may test negative for mycoplasma, endotoxin and/or microbial contamination.

### Support Matrix

A support matrix for use in accordance with the present disclosure may be made of a material to which relevant utilized cells adhere. In some embodiments, a support matrix comprises and/or is coated with an adhesive agent that facilitates and/or enables cell adherence.

In some embodiments, a support matrix supports cell proliferation.

In some embodiments, a support matrix is bioresorbable. In some such embodiments, a bioresorbable matrix may degrade over a period of hours, days, weeks or months. For example, a bioresorbable matrix may degrade within at least 24 hours, at least 7 days, at least 30 days or at least 6 months. In some embodiments, a support matrix may act as a hemostatic barrier inhibiting penetration of adjacent cells and tissues into a particular area of the body, for example, an area requiring treatment (e.g., an articular joint).

In some embodiments, a support matrix may be a gel, a solid, or a semi-solid. In some embodiments, a support matrix may be impermeable, permeable or semi-permeable (e.g., comprising pores). In some embodiments, a support matrix may be comprised of a synthetic material, a natural material, or a combination thereof.

In some embodiments, a support matrix may have a structure that comprises a membrane, microbead, fleece, thread, gel or combination thereof.

In some embodiments, a support matrix may be or comprise biological material generated by cells: in some such embodiments, a biological material may be generated by cells in culture. Alternatively, in some such embodiments, a biological material may be generated by cells in tissue (e.g., in vivo). In some embodiments, such biological material may be generated by cells that are allogeneic to a subject who will receive treatment as described herein.

In some embodiments, a support matrix may be or comprise collagen. For example, a support matrix may be or comprise type I collagen, type II collagen, type III collagen, or a combination thereof (e.g., may include a combination of type I collagen and type II collagen, or may include a combination of type I collagen and type III collagen). In some embodiments, a support matrix is comprised of primarily type I collagen on a first side and type III collagen on a second side. In some embodiments, a first side of a support matrix comprising type I collagen is a smooth surface. In some embodiments, a second side of a support matrix comprising type III collagen is a rough surface. In some embodiments, a rough surface of a support matrix is suitable for cell seeding. In some embodiments, a smooth surface of a support matrix is suitable to contact a joint surface.

In some embodiments, some or all collagen in a support matrix for use in accordance with the present disclosure may be cross-linked: in some embodiments, it may be uncross-linked.

In some embodiments, collagen utilized in accordance with the present disclosure may be derived from an animal such as a pig. In some embodiments, collagen may be derived from the peritoneum of a pig.

In some particular embodiments as described herein, a support matrix comprises a combination of type I and type III porcine collagen.

In some embodiments, cells (e.g., chondrocytes) seeded onto and/or cultured on a support matrix as described herein may produce one or more extracellular matrix proteins (e.g., collagen) that interact with and/or become incorporated into, a support matrix.

In some embodiments, a support matrix may include proteins, polypeptides, hyaluronic acid) and/or polymers (e.g., elastin, fibrin, laminin, fibronectin). In some embodiments, a support matrix may be cell-free.

In some embodiments, a support matrix may have a surface area, size, shape, and/or dimension appropriate for treatment of a particular chondral or osteochondral defect, lesion or injury. In some embodiments, a support matrix may be provided in a form (e.g., a sheet form) that is readily shaped (e.g., by folding, cutting, trimming etc.) for administration to a particular chondral or osteochondral defect.

In some embodiments, a surface area of a support matrix may be at most about 10 cm², 5 cm², 4 cm², 3 cm², 2 cm², 1 cm² or smaller. In some embodiments, a support matrix may have a surface area of about 2 cm². In some embodiments, a support matrix may have a surface area of about 3 cm². In some embodiments, a support matrix may have a surface area of about 4 cm². A dimension of a support matrix may be any dimension necessary to achieve a desired surface area suitable for treating a chondral and/or osteochondral defect. For example, dimensions of a 5 cm² support matrix may be about 1 cm×5 cm, 2 cm×2.5 cm, 3 cm×1.7 cm, or 4 cm×1.3 cm. In some embodiments, a surface area of a support matrix (e.g., collagen membrane) may be about 5 cm² with dimensions of about 1 cm×5 cm. In some embodiments, a surface area of a support matrix (e.g., collagen membrane) may be about 2 cm² with dimensions of about 2×1 cm². In some embodiments, the largest dimension of a support matrix does not exceed about 5 cm at its maximum length. In some embodiments, the largest dimension of a support matrix does not exceed about 10 cm at its maximum length. In some embodiments, the support matrix has an irregular shape.

### Cells Seeded on Support Matrix

Among other things, the present disclosure utilizes compositions comprising cultured cells (e.g., chondrocytes) seeded onto a support matrix (e.g., collagen membrane).

Typically, cells that have been cultured for a period of time (e.g., 3 days to 5 weeks) may be present on or in a support matrix. In some embodiments, cells seeded onto a support matrix may be adherent. In some embodiments, cells may be adherent to a support matrix to an extent that they do not wash off a matrix during subsequent cell culturing steps, are not displaced from a matrix during transport, and/or are not displaced from a matrix during a surgical procedure to implant a matrix.

Among other things, in some embodiments, the present disclosure utilizes cell-seeded support matrices in which a significant percentage of cells are viable; such high viability of cells present on a cell-seeded matrix can materially improve, and may be required for, successful treatment of a particular lesion or defect. In some embodiments, at least 70%, 75%, 80%, 85%, 90%, 95%, 98% or more of cells present on a cell-seeded matrix are viable. In some embodiments, at least 90% of chondrocytes present on a cell seed matrix are viable.

In some embodiments, cells seeded onto a cell-seeded support matrix are viable for at least about 1 day, 2 days, 3 days, 4, days, 5 days, 6 days, 7 days, 8 days, 9 days, 10 days, 11 days, 12 days, 13 days, 2 weeks, 3 weeks or more. In some embodiments, cells seeded onto a support matrix divide. In some embodiments, a cell-seeded support matrix is stored at about 4° C. to about 37° C.

In some embodiments, a cell-seeded support matrix comprises at least 250,000 cells/cm², 300,000 cells/cm², 400,000 cells/cm², 500,000 cells/cm², 600,000 cells/cm², 700,000 cells/cm², 800,000 cells/cm², 900,000 cells/cm², 1,000,000 cells/cm², or more. In some embodiments, a cell-seeded matrix comprising greater than 250,000 cells/cm², 300,000 cells/cm², 400,000 cells/cm², 500,000 cells/cm², 600,000 cells/cm², 700,000 cells/cm², 800,000 cells/cm², 900,000 cells/cm², 1,000,000 cells/cm² or more is suitable for implant into a subject.

In some embodiments, a cell-seeded support matrix comprises at least 5×10⁶, 7.5×10⁶, 1.0×10⁷, 1.5×10⁷, 2.0×10⁷, 2.5×10⁷, 3.0×10⁷ or more cells. In some embodiments, a 20 cm² porcine type I and type III collagen membrane comprises about 1.0×10⁷ chondrocytes to about 2.0×10⁷ chondrocytes. In some embodiments, a 14.5 cm² porcine type I and type III collagen membrane comprises about 7.5×10⁶ chondrocytes to about 1.5×10⁷ chondrocytes.

In some embodiments, a cell-seeded support matrix may comprise medium (e.g., DMEM) and supplements (e.g., fetal bovine serum, antibiotic). In some embodiments, medium comprises about 7%, about 8%, about 9%, about 10%, about 11% fetal bovine serum. In some embodiments, medium may be supplemented with 8.9%+/−0.2% fetal bovine serum and gentamicin.

In some embodiments, a cell-seeded support matrix may have a surface area of at most about 20 cm², 10 cm², 5 cm², 4 cm², 3 cm², 2 cm², 1 cm² or smaller. In some embodiments, a cell-seeded support matrix may have a surface area of about 2 cm². In some embodiments, a cell-seeded support matrix may have a surface area of about 3 cm². In some embodiments, a cell-seeded support matrix may have a surface area of about 4 cm². In some embodiments, a cell-seeded support matrix may have a surface area of about 5 cm². In some embodiments, the largest dimension of a cell-seeded support matrix does not exceed about 5 cm at its maximum length. In some embodiments, the largest dimension of a cell-seeded support matrix does not exceed about 10 cm at its maximum length. In some embodiments, a cell-seeded support matrix may be trimmed, shaped, cut, molded or formed and corresponds to a shape of a defect, lesion, and/or injury in need of treatment. In some embodiments, a cell-seeded support matrix is of an irregular shape.

In some embodiments, a cell-seeded support matrix may be substantially free of components used during preparation of a source cell preparation of during expansion of chondrocytes (e.g., fetal bovine serum albumin, fetal bovine serum and/or horse serum). For example, in some embodiments, a cell-seeded support matrix utilized herein comprises less than 10 μg/ml, 5 μg/ml, 4 μg/ml, 3 μg/ml, 2 μg/ml, 1 μg/ml, 0.05 μg/ml fetal bovine serum albumin. In some embodiments, a cell-seeded support matrix may be substantially free of mycoplasma, endotoxin, and/or microbial (e.g., aerobic microbe(s), anaerobic microbes(s) and/or fungi) contamination.

In some embodiments, a cell-seeded support matrix composition, prepared and/or utilized in accordance with the present disclosure, comprises a biocompatible adhesive or glue. In some embodiments, a least a portion of a cell-seeded matrix may be coated with a biocompatible adhesive or glue. In some embodiments, a biocompatible adhesive or glue may form a layer over cells on a support matrix. In some embodiments, a biocompatible adhesive or glue may form a layer under cells on a support matrix. In some embodiments, a cell-seeded support matrix comprises multiple layers of biocompatible adhesive or glue and cells. In some embodiments, a biocompatible adhesive or glue may be impregnated within a support matrix.

In some embodiments, the present disclosure utilizes cells and glue, and/or adhesive, combined together in a mixture of one or more alternating layers of cells and glue, and/or adhesive, on a surface or edge of a support matrix.

In some embodiments, biocompatible adhesives or glues used in compositions of the disclosure may include an organic fibrin glue (e.g., Tisseel®, fibrin based adhesive available from Baxter, Austria) or a fibrin glue prepared during surgery using autologous blood.

### Cell Sheets

Among other things, the present disclosure utilizes compositions comprising cultured cells (e.g., chondrocytes) formed into a sheet (i.e., a cell sheet).

In some embodiments, a cell sheet comprises cells in their natural extracellular matrix (ECM). In some embodiments, a cell sheet comprises chondrocytes in their natural ECM. In some embodiments, a natural ECM comprises collagen, proteoglycans, hyaluronic acid, and/or chondroitin sulfate.

In some embodiments, a cell sheet comprises a confluent cell monolayer, the confluent cells being in their natural extracellular matrix.

### Injuries and Sites

In some embodiments, the present disclosure contemplates use of cells (e.g., chondrocytes) seeded and grown on a support matrix (e.g., collagen membrane) to treat/repair cartilage defects, lesions, and/or injuries in a subject. In some embodiments, cartilage defects, lesions, and/or injuries may be located in an articulating joint (for example, knee, ankle, elbow, shoulder, hip, or wrist) of a subject. In some embodiments, a defect in a medial femoral condyle, a lateral femoral condyle, a patella, or a trochlea of a subject may be treated using technologies of the present disclosure.

Types of injuries that can lead to a cartilage defect treatable using the technologies of the present disclosure may include but are not limited to those caused by chronic and/or repetitive actions, prolonged strenuous physical activity, and trauma. Some examples of chronic and/or repetitive movements include but are not limited to walking, running, cycling, climbing, and other movements performed during exercise. Some examples of prolonged strenuous activity include but are not limited to lifting heavy objects and other forms of physical labor. Some examples of trauma include but are not limited to falls, collisions, and sports-related injuries.

In some embodiments, a subject who may be treated is an adult human. In some embodiments, a subject who may be treated is under the age of 18. In some embodiments, a subject who may be treated is a human between 10 and 17 years of age: in some such embodiments, a subject does not have an open growth plate. In some embodiments, a subject displays symptoms of a cartilage defect. In some embodiments, symptoms of a cartilage defect may include joint pain, joint swelling, and/or changes in joint flexibility and/or movement. In some embodiments, a subject may be asymptomatic.

### Methods

The present disclosure provides technologies for the delivery of compositions to a surgical site, the compositions comprising cells, which compositions may be useful, for example, for treatment of chondral and/or osteochondral lesions (e.g., focal lesions in the load bearing region of a knee's articular cartilage).

In some embodiments, the present disclosure provides technologies that permit and/or achieve treatment of clinically significant chondral and/or osteochondral lesions, defects, injuries and/or trauma. In some embodiments, treatment comprises tissue repair and/or regeneration.

In some embodiments, compositions comprising chondrocytes may be implanted into a subject at or near a site of a lesion, defect, injury and/or trauma, for example, at or near an articular surface, using arthroscopic methods. Articular surfaces that may be treated using the methods and compositions of the present disclosure include articular surfaces of, for example, a knee, ankle, wrist, hip, elbow, and/or shoulder.

### Open Administration

Traditionally, procedures involving the implantation of a cell-seeded support matrix at a site of a defect, lesion and/or injury, have been performed under open surgical conditions requiring a large incision adjacent to the site. The implantation of a cell-seeded support matrix has traditionally been performed via an arthrotomy adjacent to the site under sterile conditions. In many of these procedures, a mini-arthrotomy is used. Mini-arthrotomy to repair knee defects (e.g., lesions on the condyle and patella) generally requires an incision with a length ranging from about 6 cm to about 10 cm. Open surgical procedures such as arthrotomy are typically used because they provide surgeons the ability to visualize and measure defects, as well as to physically manipulate the implant near the defect with relative ease.

The present disclosure appreciates various disadvantages of open surgical methods, including those traditionally used in the MACI procedure, when compared to minimally-invasive methods such as arthroscopy. For example, the relatively large incisions required to perform many open surgical techniques, including those traditionally used in the MACI procedure, present an increased risk of infection, an increased risk of significant scarring, longer recovery times, and increased pain severity, relative to the same metrics following minimally invasive procedures such as arthroscopic implantation.

In such open surgical procedures, typically, an incision may be made to allow access to a joint to be surgically treated, such that the joint and its internal tissue (e.g., cartilage) are exposed and visible to a physician performing the procedure. Typically, preparation of the surgical site may include washing the site and removing damaged cartilage from the site. Typically, a cell-seeded support matrix is placed with cells facing (e.g., in contact with) a surface to be treated. In some such procedures, a cell-seeded support matrix is implanted into, and/or over, a site of a lesion, defect and/or injury. A cell-seeded support matrix may be provided in a form (e.g., a sheet form) that is readily shaped (e.g., by folding, cutting, trimming etc.) for administration to a chondral or osteochondral defect. In some procedures, a cell-seeded support matrix is shaped into a form that uniquely fits or adheres to a chondral or osteochondral defect of a subject. The cell-seeded support matrix is typically secured in the site using a fixation method, for example, fibrin glue fixation. The site may then be closed, the cell-seeded matrix remaining in the site.

### Arthroscopic Delivery

Arthroscopy (also called arthroscopic or keyhole surgery) is a minimally invasive surgical procedure on a joint in which an examination and sometimes treatment of damage is performed using an arthroscope, an endoscope that is inserted into the joint through a small incision. Arthroscopic procedures can be performed under numerous surgical scenarios, including but not limited to ACL reconstruction, meniscus reconstruction, and cartilage repair.

Arthroscopic surgery has become a preferred surgical method due at least in part to its positive impact on patient health outcomes, including but not limited to minimal soft tissue trauma, low post-operative pain, fast healing times, and low infection rates. Many of the surgical repairs that benefit from MACI are at sites that are accessible using arthroscopic surgical methods. The present disclosure provides technologies that permit the MACI procedure via an arthroscopic delivery method.

A critical advantage of arthroscopic surgery over traditional open surgery is that a joint does not have to be opened and fully exposed during the surgical procedure. In some arthroscopic procedures performed on the knee, only around two small incisions are made: one for the arthroscope and at least one for the surgical instruments to be used in the knee cavity. This may reduce recovery time and may increase the rate of success due to less trauma to connective tissue, as compared to traditional open surgical procedures. In recent years, arthroscopy has gained popularity owing at least in part to evidence of faster recovery times with less scarring, due at least in part to smaller incisions. Irrigation fluid (most commonly normal saline) may be used to distend the joint and make a surgical space.

In traditional arthroscopic procedures, the surgical instruments used are smaller than traditional surgical instruments. Surgeons view the joint area on a video monitor, and can diagnose and repair defects in joint tissue. It is possible to perform an arthroscopic examination of almost every joint. Arthroscopic procedures are most commonly performed on the knee, shoulder, elbow, wrist, ankle, foot, and hip.

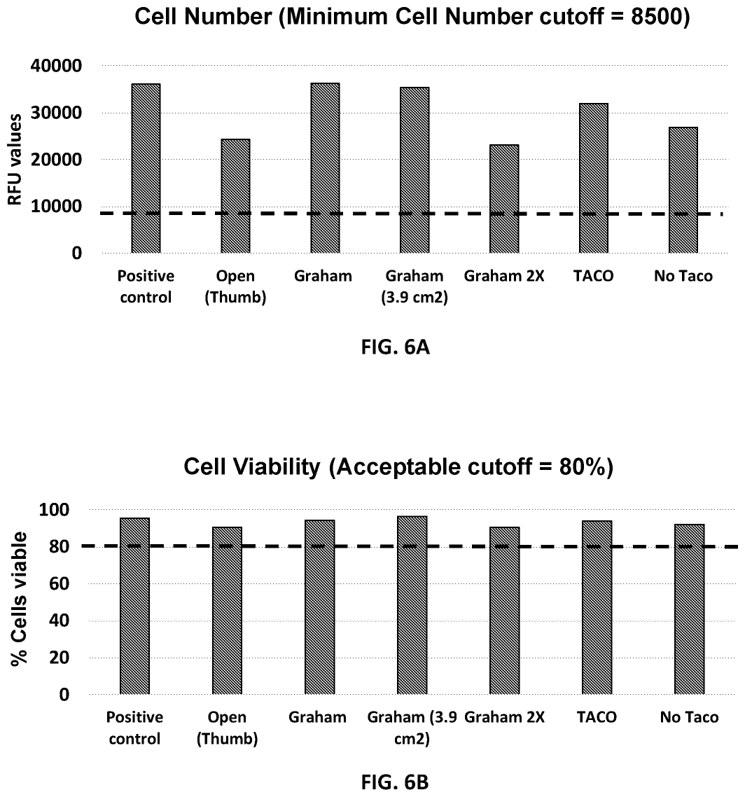

The present disclosure appreciates the source of a challenge encountered in delivery of cell-seeded matrix compositions via arthroscopic procedures. For example, among other things, the present disclosure identifies that, absent technologies described herein, it may be difficult or impossible to maintain appropriate (e.g., sufficient) levels of cell viability. Among other things, the present disclosure provides solutions. For example, the present disclosure provides technologies that are demonstrated herein to achieve arthroscopic delivery while maintaining cell viability (e.g., as assessed by one or more parameters described herein) reasonably comparable to those found with certain open surgical methods. The present disclosure describes certain surprising and unexpected results (see, for example, Example 6 and FIG. 6) that provided technologies achieve, including cell viability levels that can that meet, or even exceed, those obtained by certain open surgical delivery methods.

In some embodiments, at least two incisions may be made adjacent to the location of a defect to be treated arthroscopically. In some embodiments, incisions may have a length in a range from about 1 cm to about 2 cm. In some embodiments, at least one incision may be made to accommodate the insertion of an arthroscope. In some embodiments, at least one incision may be made to accommodate the insertion of a cannula. In some embodiments, at least 2, at least 3, or at least 4 incisions may be made. In some embodiments, at least 2 incisions may be made, each to accommodate the insertion of a cannula.

In some embodiments, the size and/or shape of a defect may be determined prior to arthroscopic implantation of a cell-seeded matrix to a defect. In some embodiments, the size and/or shape of a defect may be determined by comparing the size and/or shape of the defect to the size and/or shape of a surgical tool (e.g., a surgical probe or surgical measuring device). In some embodiments, the size and/or shape of a defect may be determined by iteratively comparing the size and/or shape of the defect to the size and/or shape of a piece of templating material. In some embodiments, a piece of templating material may be shaped to match the size and shape of a defect. In some embodiments, templating material used to approximate the size and shape of a defect in a surgical site may be used as a template for shaping an implant to be delivered to the defect in the site. In some embodiments, templating material used to approximate the size and shape of a defect in a surgical site may be used as backing material for an implant to be delivered to the defect in the surgical site. In some embodiments, a piece of templating material comprises sterile paper, sterile aluminum foil, a sterile bandage, or another sterile material that is flexible and may be manipulated.

In some embodiments, a cell-seeded support matrix may be implanted at a site of a defect, lesion and/or injury using an arthroscopic technique. In some embodiments, when a cell-seeded support matrix is implanted at a site of a defect, lesion, and/or injury using an arthroscopic technique, a matrix may be placed with cells facing (e.g., in contact with) a surface to be treated. In some embodiments, a cell-seeded support matrix may be arthroscopically implanted into, and/or over, a site of a lesion, defect, and/or injury. In some embodiments, a cell-seeded support matrix may be provided in a form (e.g., a sheet form) that is readily shaped (e.g., by folding, cutting, trimming etc.) for arthroscopic administration to a chondral or osteochondral defect. In some embodiments, a cell-seeded support matrix may be shaped into a form that uniquely fits or adheres to a subject's chondral or osteochondral defect, prior to arthroscopic implantation.

In some embodiments, one or more cell-seeded support matrices may be arthroscopically implanted to treat a region comprising a defect, lesion, and/or injury. In some embodiments, 1, 2, 3, 4, or 5 or more cell-seeded support matrices may be arthroscopically implanted in a region that includes a defect, lesion and/or injury. In some embodiments, more than one cell-seeded support matrix may be layered into, or over, a defect, lesion, and/or injury via arthroscopy. In some embodiments, more than one cell-seeded support matrix may be tiled into, or over, a defect, lesion, and/or injury via arthroscopy, thereby expanding the possible target treatment area that can be treated via the present embodiments.

In some embodiments, a single matrix may be utilized to treat multiple defects via arthroscopy. In some embodiments, a plurality of defects may be treated, each with a different matrix, at least some of which are delivered via arthroscopy. In some embodiments, one or more defects may be treated with a plurality of individual matrices via arthroscopy.

In some embodiments, following treatment comprising arthroscopic delivery of a composition of the present disclosure, a treated region (e.g., an articular joint) may be evaluated using a screening method (e.g., magnetic resonance imaging). In some embodiments, a treated region may be evaluated for filling, repair, and/or healing of a defect, lesion, and/or injury.

In some embodiments, a cell-seeded support matrix may be arthroscopically implanted at a site of a defect, lesion, and/or injury using a cannula. In some embodiments, a cannula may have an inner diameter of about at least 5 mm, at least 6 mm, at least 7 mm, at least 8 mm, at least 9 mm, or at least 10 mm. In some embodiments, a cannula may have an inner diameter from about 8 mm to about 9 mm. In some embodiments, a cannula may have an inner diameter greater than 10 mm. In some embodiments, a cannula may have an inner diameter from about 15 mm to about 20 mm. In some embodiments, the cannula may have a length that is about 2 cm, about 3 cm, about 4 cm, about 5 cm, about 6 cm, about 7 cm, about 8 cm, about 9 cm, or about 10 cm or longer. In some embodiments, a cannula may have a length that is in a range from about 2 cm to about 10 cm. In some embodiments, a cannula has a length that is about 4.5 cm. In some embodiments, the length of a cannula may depend on the location of the site of the defect to be treated. For example, a cannula used to treat a hip defect may have a length that is about 12 cm to about 20 cm. In some embodiments, a cannula used to treat a hip defect may have a length that is about 16.5 cm. In some embodiments, a cannula used to treat a shoulder defect may have a length that is about 12 cm to about 20 cm. In some embodiments, a cannula used to treat a shoulder defect may have a length that is about 16.5 cm. As an additional example, a cannula used to treat a knee defect may have a length that is in a range from about 2 cm to about 7 cm. In some embodiments, a cannula used to treat a knee defect may have a length that is about 4.5 cm.

In some embodiments, a cannula may be composed of a material comprising plastic. In some embodiments, a cannula may be composed of a material comprising metal. In some embodiments, a cannula may be composed of a material selected from the group consisting of plastics, metals, rubber, silicone, fiber glass, and combinations thereof (for example, composite materials).

In some embodiments, one end of a cannula may be truncated at an angle. In some embodiments, one end of a cannula may be truncated at an angle of up to about 30 degrees from a longitudinal axis of the cannula, or about 60 degrees from a perpendicular axis of the cannula. In some embodiments, one end of a cannula may be truncated at an angle of up to about 45 degrees from a longitudinal axis of the cannula. In some embodiments, one end of a cannula may be truncated at an angle from about 15 degrees to about 45 degrees from a longitudinal axis of a cannula, or about 45 degrees to about 75 degrees from a perpendicular axis of the cannula.

In some embodiments, a cell-seeded support matrix may be arthroscopically delivered to a surgical site by grasping one edge of a cell-seeded support matrix with a surgical grasper, and pushing the cell-seeded support matrix into a cannula positioned in a surgical site. In some embodiments, a cell-seeded support matrix may be arthroscopically delivered to a surgical site by folding the cell-seeded support matrix in half, grasping the folded support matrix where its edges meet using a surgical grasper, and pushing the folded cell-seeded support matrix into a cannula positioned in a surgical site. In some embodiments, a cell-seeded support matrix may traverse the entire length of a cannula.

In some embodiments, a surgical grasper may be a small joint grasper. In some embodiments, a surgical grasper comprises atraumatic jaws. In some embodiments, a cell-seeded support matrix comprising a cell seeded surface and a non-cell-seeded surface may be loaded into a cannula, for example, a delivery cannula, by pulling the matrix into the distal end of a cannula using a surgical grasper such that the cell-seeded surface faces the cannula lumen and its non-cell-seeded surface contacts the cannula interior wall. In some embodiments, a cannula comprising a support matrix loaded into its distal end may be positioned relative to a surgical site so that the distal end is toward the surgical site. In some embodiments, a matrix loaded into the distal end of a cannula positioned toward a surgical site may be pushed out of the distal end of the cannula and into the site so that the cell-seeded surface contacts tissue in the site. In some embodiments, a cell-seeded support matrix may be delivered into a surgical site so that the cell-seeded support matrix does not traverse the entirety of the cannula during the delivery.

In some embodiments, a cell-seeded support matrix may be arthroscopically implanted at a site of a defect, lesion, and/or injury using at least 2, at least 3, at least 4, or at least 5 or more cannulas. In some embodiments, a cell-seeded support matrix may be arthroscopically implanted at a site of a defect, lesion, and/or injury using a loading cannula and a delivering cannula. In some embodiments, a cell-seeded support matrix may be arthroscopically implanted at a site of a defect by loading a cell-seeded support matrix into the distal end of a loading cannula by pulling the cell-seeded support matrix into the loading cannula using a surgical grasper, positioning a delivering cannula into a surgical site, connecting the distal end of the loading cannula to the end of the delivering cannula that is not in the surgical site, and pushing the cell-seeded support matrix through the entire length of the delivering cannula and into the surgical site.

In some embodiments, after a cell-seeded support matrix is implanted into a defect, lesion, and/or injury, a covering patch may be secured using e.g., a biocompatible adhesive, sealant, or suture. In some embodiments, a covering patch may serve to cover an area to prevent infiltration of undesirable cells and/or biological factors (e.g., fibroblasts, macrophages) from surrounding tissue into an area to be treated. In some embodiments, a covering patch comprises any support matrices described herein, and/or may include hyaluronic acid, fibrin, and/or polylactic acid. In some embodiments, a covering patch may be cell-free and resorbable. In some embodiments, a covering patch may be semi-permeable.

In some embodiments, biocompatible adhesives or glues used to secure a covering patch may include an organic fibrin glue or sealant (e.g., Tisseel®, fibrin based adhesive available from Baxter, Austria) or a fibrin glue prepared during surgery using autologous blood.

In some embodiments, a biocompatible adhesive or glue may be applied to a defect prior to placement of a cell-seeded support matrix over, or into, a defect. In some embodiments, a biocompatible adhesive or glue may be applied to a cell-seeded support matrix prior to placement over, or into, a defect. In some embodiments, a biocompatible adhesive or glue may be applied to a periphery of an implant.

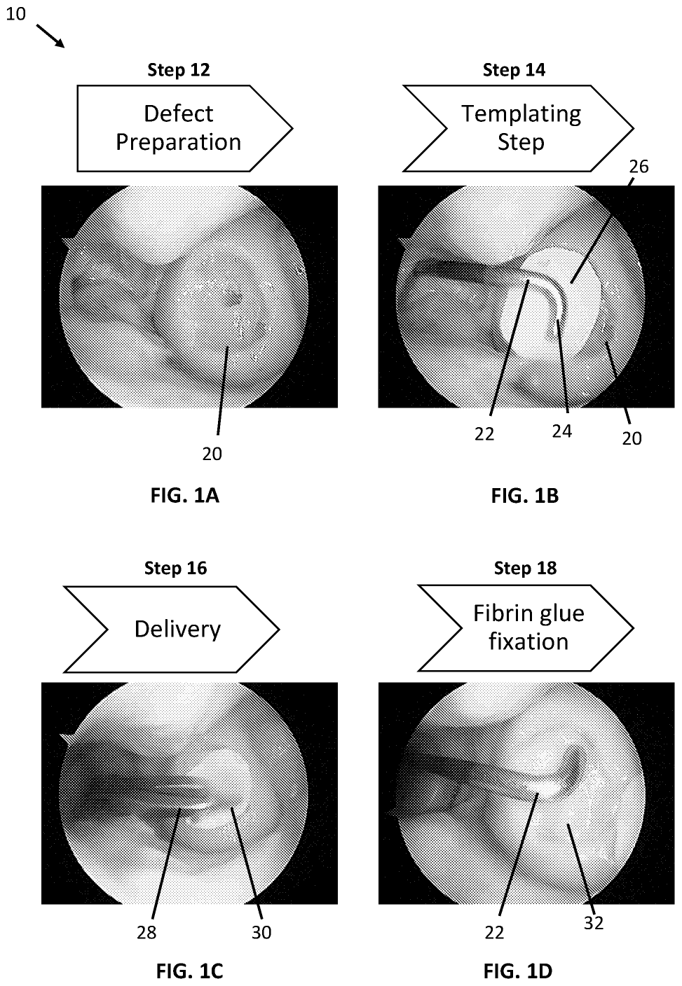

FIG. 1 illustrates an arthroscopic delivery method 10, according to aspects of the present embodiments. The method 10 may generally include the steps of preparing the defect site (at step 12), preparing a template (step 14), delivering a treatment to the defect site (step 16) and applying Fibrin glue (or other suitable glue) in a fixation step (step 18). At step 12, the method 10 may include preparing the defect site 20, shown in FIG. 1A. Preparing the defect site 20 may include using curettes to remove all damaged and fibrous tissue from the defect site. Preparing the defect site 20 may also include creating clean vertical borders at the defect edges. Excision should remove the calcified cartilage layer without penetrating the subchondral bone. Care should be taken to avoid penetrating the subchondral bone. At step 14, the method 10 may include a templating step which may include delivering a template material 26 to the defect site 20 to measure the geometry and/or dimensions of the defect site 20 compared with the geometry of the template material 26. (The defect site 20 is partially visible behind the template material 26, as shown in FIG. 1B). The template material 26 may be delivered to the defect site 20 via a surgical grasper 28, which may include a set of jaws 24 for holding the templating material 26. A probe 22 may be used to facilitate approximating the size and shape of the defect site 20.

Referring still to FIG. 1, after the templating step 14 is complete, a cell-seeded support matrix 30 may be prepared by seeding a support matrix that includes dimensions and/or the general geometric shape as the defect site 20, as measured by the template material. The cell-seeded support matrix 30 may then be delivered (at step 16) to the defect site via a surgical tool 28. (The defect site 20 is not visible in the view of FIG. 1C because it is behind the cell-seeded support matrix 30). During the delivery step 16, cells from the cell-seeded support matrix 30 are brought into direct contact with the defect site 20. At step 18, the method 10 may include applying a Fibrin glue 32 (or other suitable material) to the defect site 20 using a probe 22.

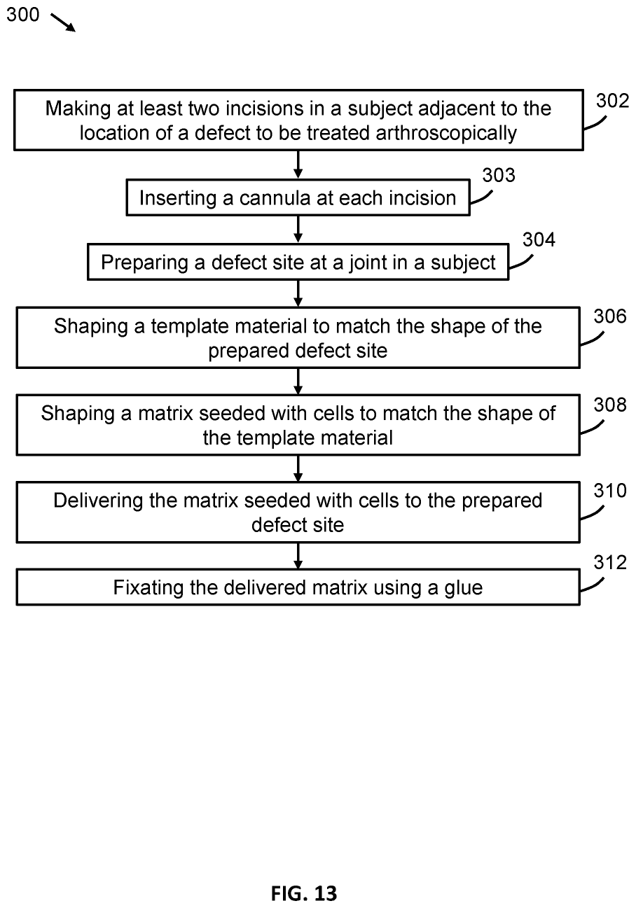

The methods described above for arthroscopic surgery are summarized in FIG. 13, which depicts a flowchart of an arthroscopic surgical method 300 to implant a cell-seeded matrix implant at a defect site, according to aspects of the present embodiments. The method 300 may generally include the steps of making at least two incisions in a subject adjacent to the location of a defect to be treated arthroscopically (step 302); preparing a defect site at a joint in a subject (step 304); templating a template material to match the shape of the prepared defect site (step 306); shaping a matrix seeded with cells to match the shape of the template material (step 308); delivering the matrix seeded with cells to the prepared defect site (step 310); and fixating the delivered matrix using a glue (step 312). In some embodiments, at step 303, the method 300 may also include inserting a cannula at each incision prior to preparing the defect site at a joint in the subject.

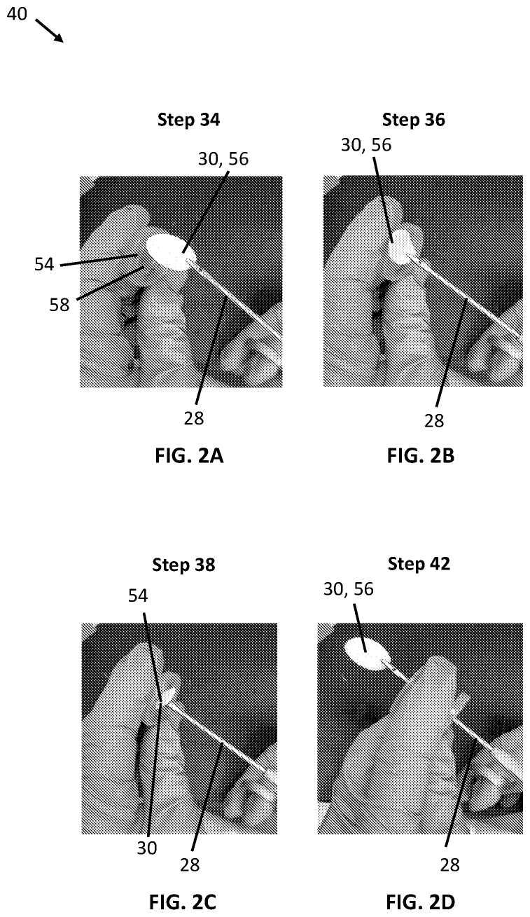

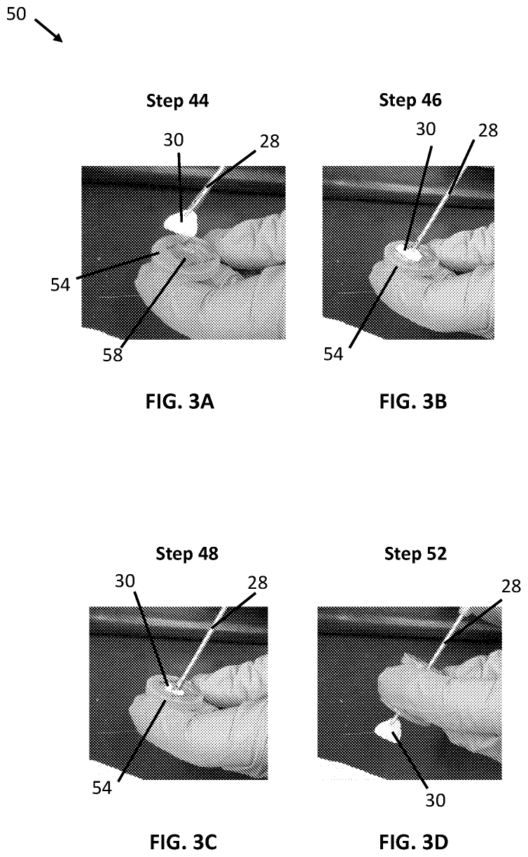

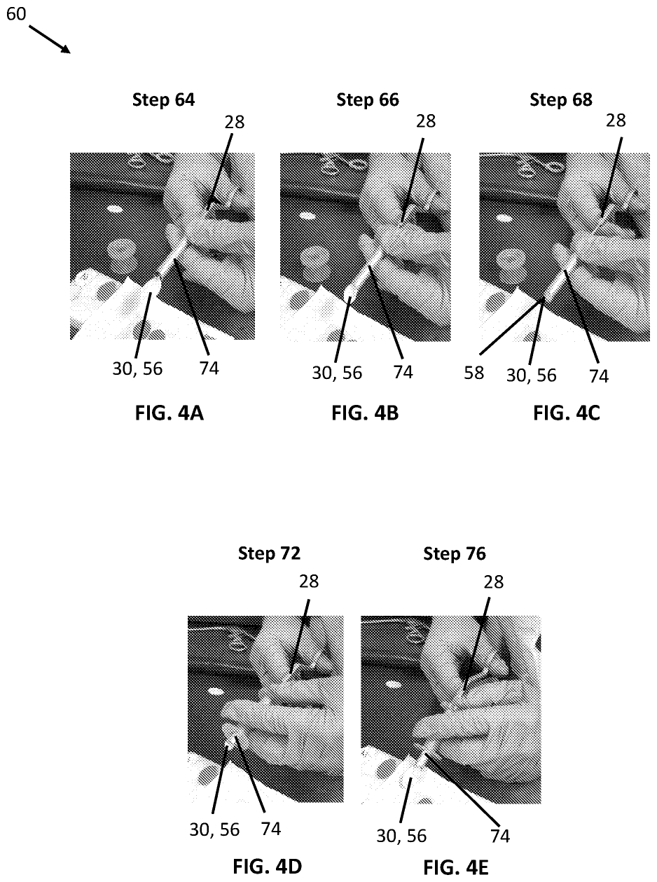

FIG. 2 illustrates an arthroscopic delivery method 40, according to aspects of the present embodiments. The method 40 illustrated in FIG. 2 may generally be referred to as an “unfolded” method because it uses a support matrix that is not folded prior to insertion. At step 34, the method may include grabbing the cell-seeded support matrix 30 using a surgical grasper (tool 28). The method 40 illustrated in FIG. 2 may include the use of a cannula 54, which would be installed within a patient in practice, but is shown ex vivo in FIG. 2 (as well as FIG. 3 and FIG. 4) for illustration purposes. The cannula 54 may include a lumen 58 disposed therein. The lumen 58 may act as a center borehole within the cannula 54 through which surgical tool 28 and/or the cell-seeded support matrix 30 may be disposed. The cell-seeded support matrix 30 may include a cell-seeded surface 56, visible in the illustration of FIG. 2A. In some embodiments, the reverse side of the cell-seeded surface 56 would also be seeded with cells, while in other embodiments, the reverse side of the cell-seeded surface 56 would be unseeded. At step 36, the method 40 may include bringing the cell-seeded support matrix 30 into contact with the cannula 54 such that an unseeded surface of the cell-seeded support matrix 30 contacts the cannula 54 (and thereby protecting the cell-seeded surface 56 from coming into contact with the cannula 54), as shown in FIG. 2B.

Referring still to FIG. 2, at step 38, the method may include pushing the cell-seeded support matrix 30 into the lumen 58, as shown in FIG. 2C. (In the illustration of FIG. 2C, the lumen 58 is not visible because it is behind the cell-seeded support matrix 30.) As the cell-seeded support matrix 30 is pushed into the cannula 54, it partially folds such that the cell-seeded surface 56 is on the inside and the unseeded surface is on the outside. At step 42, the method 40 may include using the surgical tool 28 to push the cell-seeded support matrix 30 through to the opposite side of the cannula 54, which will be at or proximate the defect site 20 in practice (in which case, the cannula 54 would be disposed within the patient).

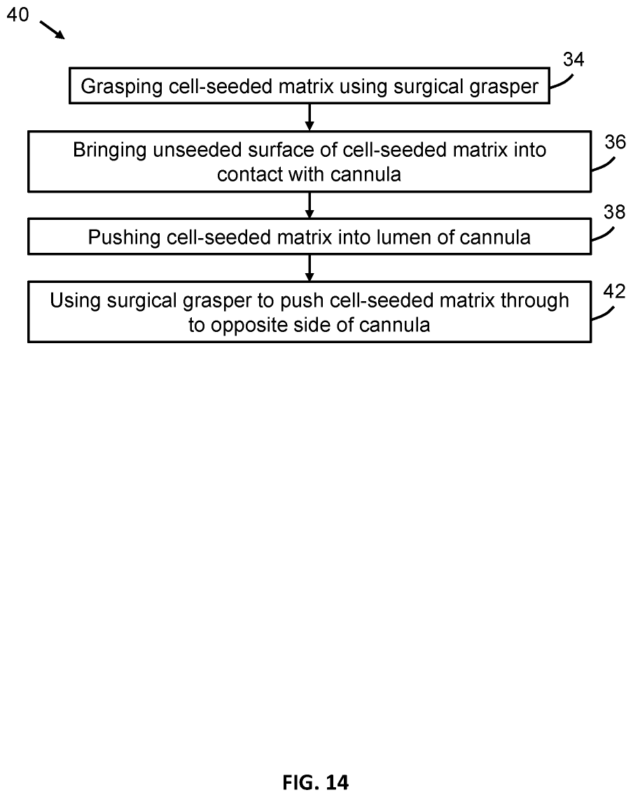

The delivery of the cell-seeded matrix by the unfolded method is summarized in FIG. 14, which depicts a flowchart of a method 40 to deliver a cell-seeded matrix by unfolded method, according to aspects of the present embodiments. The method 40 is also illustrated in the photographs in FIG. 2. The method 40 may generally include the steps of grasping a cell-seeded matrix using a surgical grasper (step 34); bringing the unseeded surface of the cell-seeded matrix into contact with the proximal opening of a cannula (step 36); pushing the cell-seeded matrix into the lumen of the cannula (step 38); and using the surgical grasper to push the cell-seeded matrix through to the distal end of the cannula (step 42).

FIG. 3 illustrates an arthroscopic delivery method 50, according to aspects of the present embodiments. The method 50 illustrated in FIG. 3 may generally be referred to as a “folded” method because it uses a support matrix that is folded prior to insertion. At step 44, the method may include folding the cell-seeded support matrix 30 in half as pictured. The cell-seeded support matrix 30 may include a cell-seeded surface. In some embodiments, the reverse side of the cell-seeded surface may also be seeded with cells, while in other embodiments, the reverse side of the cell-seeded surface may be unseeded. The cell-seeded support matrix 30 may be folded such that the cell-seeded surface is the outside surface of the folded cell-seeded support matrix 30. Alternatively or additionally, the cell-seeded support matrix 30 may be folded such that the cell-seeded surface is the inside surface of the folded cell-seeded support matrix 30. Also at step 44, the method may include grabbing the cell-seeded support matrix 30 where a first outside edge meets a second outside edge using a surgical grasper (tool 28). The method 50 illustrated in FIG. 3 may include the use of a cannula 54, which would be installed within a patient in practice, but is shown ex vivo in FIG. 3 (as well as in FIG. 2 and FIG. 4) for illustration purposes. The cannula 54 may include a lumen 58 disposed therein. The lumen 58 may act as a center borehole within the cannula 54 through which surgical tool 28 and/or the cell-seeded support matrix 30 may be disposed. At step 46, the method 50 may include bringing the folded cell-seeded support matrix 30 into contact with the cannula 54 as shown in FIG. 3B.

Referring still to FIG. 3, at step 48, the method may include pushing the folded cell-seeded support matrix 30 into the lumen 58, as shown in FIG. 3C. (In the illustration of FIG. 3C, the lumen 58 is not visible because it is behind the cell-seeded support matrix 30.) As the folded cell-seeded support matrix 30 is pushed into the cannula 54, it folds toward the surgical grasper (tool 28). At step 52, the method 50 may include using the surgical tool 28 to push the folded cell-seeded support matrix 30 through to the opposite side of the cannula 54 (as shown in FIG. 3D), which will be at or proximate the defect site 20 in practice (in which case, the cannula 54 would be disposed within the patient).

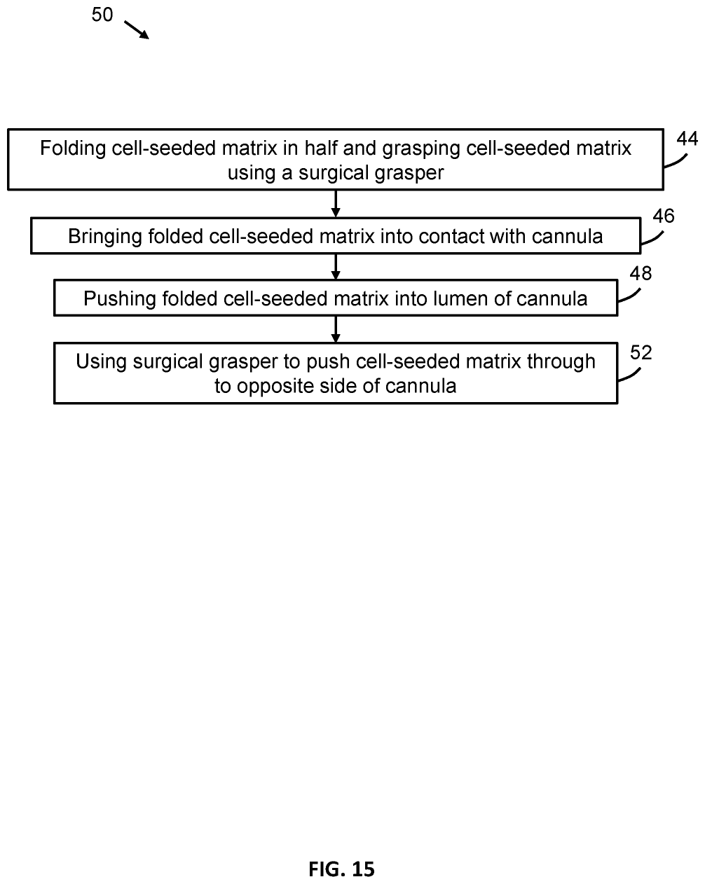

The delivery of the cell-seeded matrix by the folded method is summarized in FIG. 15 which depicts a flowchart of a method 50 to deliver a cell-seeded matrix by folded method, according to aspects of the present embodiments. The method 40 is also illustrated in the photographs in FIG. 3. The method 50 may generally include the steps of folding the cell-seeded matrix in half and grasping the cell-seeded matrix using a surgical grasper (step 44); bringing folded cell-seeded matrix into contact with the proximal opening of a cannula (step 46); pushing the folded cell-seeded matrix into the lumen of the cannula (step 48); and using the surgical grasper to push the cell-seeded matrix through to the opposite side of cannula (step 52).

FIG. 4 illustrates an arthroscopic delivery method 60, according to aspects of the present embodiments. The method 60 illustrated in FIG. 4 may generally be referred to as a “distal loading” method because it uses a support matrix that is loaded into the distal end of a cannula prior to insertion. At step 64, the method 60 may include disposing a surgical tool 28 in the lumen 58 of a cannula 74 such that the jaws of the surgical tool 28 protrude from the distal end of the cannula 74 as depicted in FIG. 4A. Also at step 64, the method may include grabbing the cell-seeded support matrix 30 using a surgical tool 28. The cell-seeded support matrix 30 may include a cell-seeded surface 56, visible in FIG. 4A. In some embodiments, the reverse side of the cell-seeded surface would also be seeded with cells, while in other embodiments, the reverse side of the cell-seeded surface would be unseeded. At step 66, the method may include pulling a cell-seeded matrix 30 into a cannula 74 using a surgical tool 28. The process of pulling the cell-seeded matrix 30 into the cannula 74 may cause the cell-seeded matrix 30 to partially fold to fit the shape of the cannula 74, as depicted in FIG. 4B. At step 68, the method may include pulling the cell-seeded matrix 30 fully into the cannula 74 such that the cell-seeded surface 56 faces the cannula lumen 58, and the reverse side of the cell-seeded matrix contacts the interior wall of the cannula 74, as shown in FIG. 4C.

Referring still to FIG. 4, at step 72, the method may include using the surgical tool 28 to push the partially folded cell-seeded support matrix 30 out of the distal end of the cannula 74, as shown in FIG. 4D. At step 76, the method may include using the surgical tool 28 to push the partially folded cell-seeded support matrix 30 out of the distal end of the cannula 74 such that the cell-seeded support matrix 30 unfolds and becomes amenable to contacting a surface, as shown in FIG. 4E. In practice, the distal end of the cannula will be at or proximate the defect site 20 (in which case, the cannula 74 would be disposed within the patient). In some embodiments, after performing step 68 and before performing step 72, the method may include connecting the cannula 74 to a second cannula (receiving cannula or delivering cannula: not shown). In some such embodiments, the method may include using the surgical tool 28 to push the partially folded cell-seeded support matrix 30 out of the distal end of the cannula 74, into the proximal end of the second cannula, through the entirety of the second cannula, out of the distal end of the second cannula. In some such embodiments, the second cannula is positioned in a surgical site in a patient prior to the step of connecting.

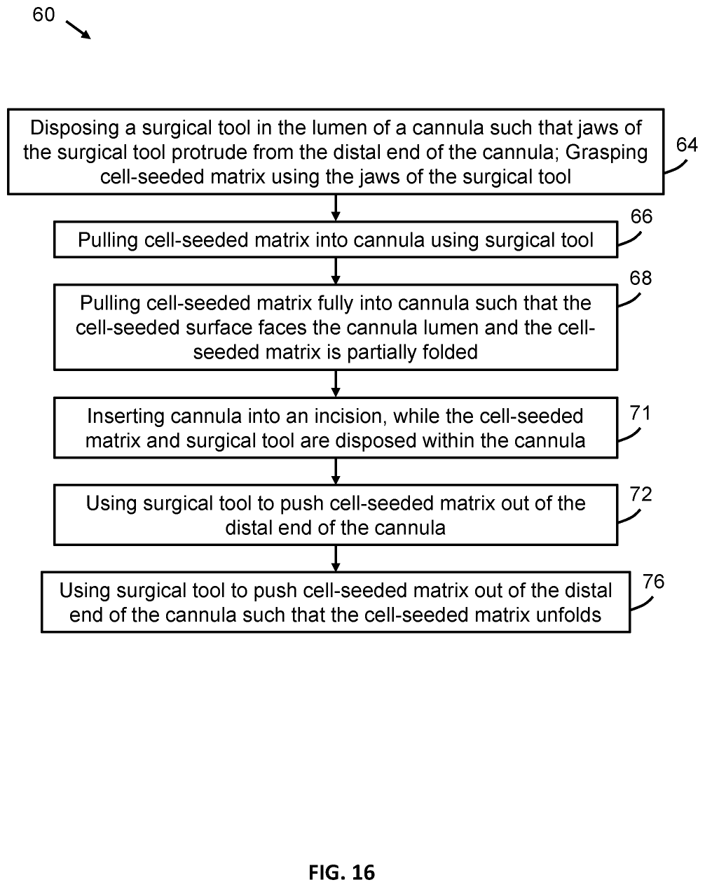

The delivery of the cell-seeded matrix by the distal loading method is summarized in FIG. 16, which depicts a flowchart of a method 60 to deliver a cell-seeded matrix by distal loading method, according to aspects of the present embodiments. The method 60 is also illustrated in the photographs in FIG. 4. The method 60 may generally include disposing a surgical tool in the lumen of a cannula such that jaws of the surgical tool protrude from the distal end of the cannula, and grasping a cell-seeded matrix using the surgical tool (step 64); pulling the cell-seeded matrix into the cannula using the surgical tool (step 66) such that the cell-seeded surface faces the cannula lumen and may be partially folded (step 68); inserting the cannula into an incision, while the cell-seeded matrix and the surgical tool are disposed within the cannula (step 71); using the surgical tool to push the cell-seeded matrix out of the distal end of the cannula (step 72); and using the surgical tool to push the cell-seeded matrix further out of the distal end of the cannula such that the cell-seeded matrix unfolds (step 76).

### Systems/Tools/Devices/Kits

Provided herein are systems, tools, devices, and kits useful for practicing the methods of the invention, which will allow for the convenient practice of the methods of the invention in a surgical setting.

In some embodiments, at least one custom device may be used to perform methods described herein.

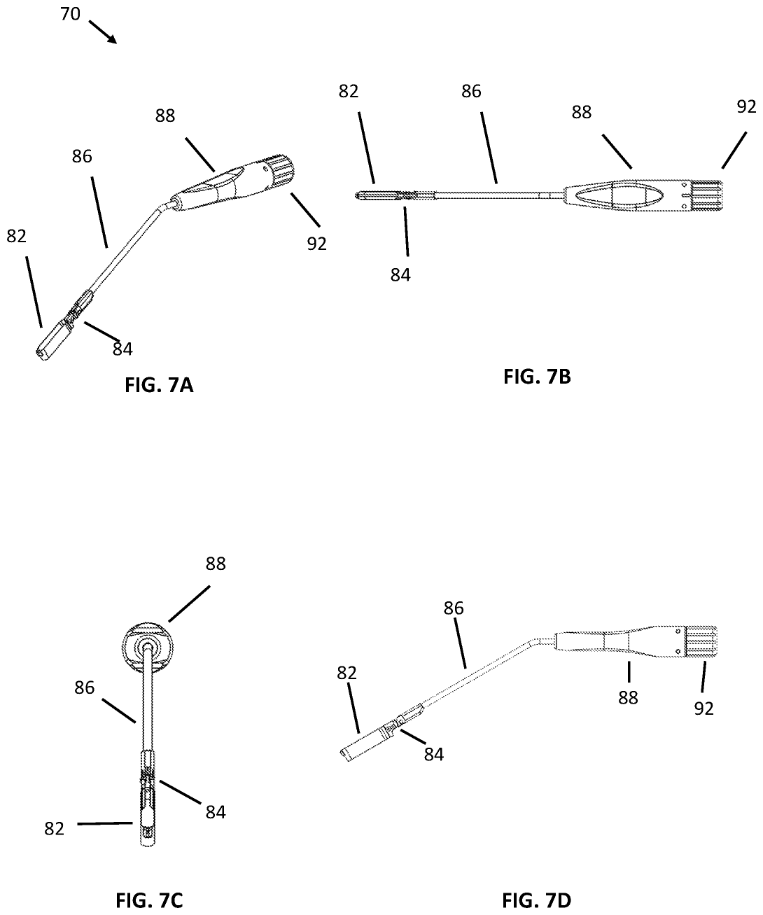

FIG. 7 illustrates multiple views of a custom device 70, according to aspects of the present embodiments. Generally, the custom device may include a handle 88, an adjustable knob (or adjusting knob) 92 coupled (for example, rotatably coupled) to the handle 88 at a proximal end, a shaft 86 coupled to a distal end of the handle 88, a movable joint 84 disposed at the distal end of the shaft 86, and an adjustable distal end 82 (for example, an adjustable cutter 82) coupled to the moveable joint 84. The shaft 86 may be coupled to the handle 88 at a fixed angle (for example, from about 5 degrees to about 50 degrees, or from about 10 degrees to about 40 degrees, or from about 15 degrees to about 30 degrees, from about 20 degrees to about 25 degrees, and/or other various sub-ranges therebetween). The adjustable distal end 82 (for example, an adjustable cutter 82) may be adjusted such that an angle between the adjustable distal end 82 and the shaft 86 changes.

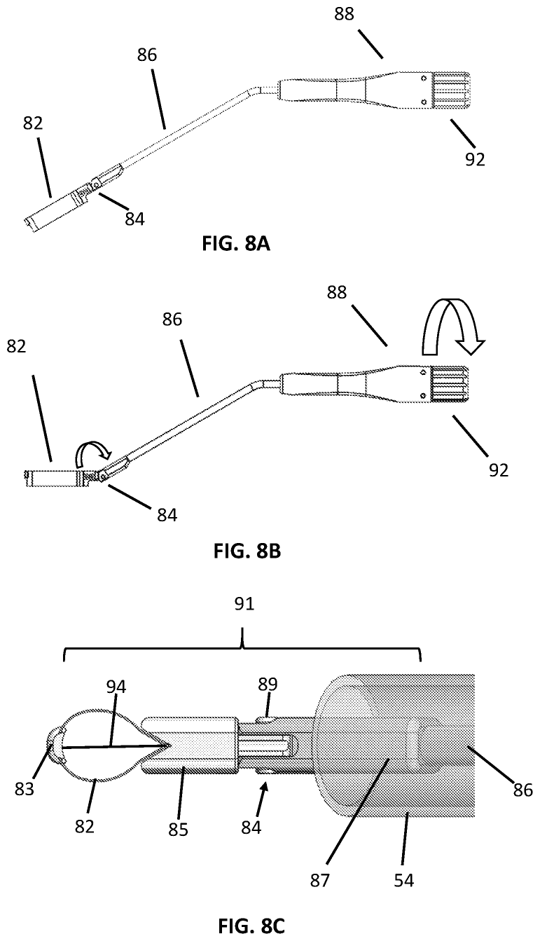

In some embodiments, a custom device may be or comprise an adjustable cutting device. In some embodiments, an adjustable cutting device may comprise an adjustable cutter 82. In some embodiments, an adjustable cutter 82 is composed of a material comprising metal. In some embodiments, an adjustable cutting device may comprise a moveable joint 84. In some embodiments, an adjustable cutting device may comprise a shaft 86. In some embodiments, a moveable joint 84 allows an adjustable cutter 82 to be oriented at an angle ranging from about 0 degrees to about 90 degrees relative to an axis parallel to a shaft 86. In some embodiments, an adjustable cutting device may comprise a handle 88. In some embodiments, an adjustable cutting device may comprise an adjusting knob 92. In some embodiments, an adjustable cutting device may comprise at least one cable. In some embodiments, an adjusting knob 92 is coupled to a cable that, upon rotation of the adjusting knob 92, exerts a force resulting in a change in an angle of a joint 84, as shown in FIG. 8. In some embodiments, an angle is a fixed angle.

Referring still to FIG. 7, the custom device 70 may be used to measure, cut, position, and/or remove tissue from a target site. The adjustable cutter 82 may initially be co-linear with the shaft 86 (as shown in each of FIGS. 7A-7D) to allow the distal end of the custom device 70 to fit through the cannula 54. Once the distal end of the custom device 70) is inserted through the cannula 54 to the target location, the angle between the adjustable cutter 82 and the shaft 86 can be adjusted such that he adjustable cutter 82 is rotated about the moveable joint 84. The rotation of the adjustable cutter 82 about the moveable joint 84 can be executed via one or more cables (not shown) extending from the adjustable cutter 82, through the moveable joint 84, shaft 86, and handle 88, to the adjusting knob 92. As the adjusting knob 92 is turned, the cable pulls the adjustable cutter 82, thereby allowing it to rotate and change the angle between the adjustable cutter 82 and shaft 86. In some embodiments, the angle to which the adjustable cutter 82 gets adjusted is a pre-determined, fixed angle (i.e., from about zero degrees and to about 180 degrees, from about zero degrees and to about 135 degrees, from about zero degrees and to about 90 degrees, from about zero degrees and to about 60 degrees, from about zero degrees and to about 50 degrees, from about zero degrees and to about 45 degrees, from about zero degrees and to about 35 degrees, and/or from about zero degrees and to about 30 degrees).

Still referring to FIG. 7, once inserted in the cannula 54 (shown in FIGS. 2-4 and 8C), the custom device 70 may be rotated from zero to 360 degrees (and sub-ranges therebetween) within the cannula 54 (for example, with the shaft 36 concentrically disposed within the cannula 54). In addition, the adjustable cutter 82 may be rotated to a desired and/or predetermined angle. By rotating the entire custom device 70 in concert with adjusting the angle between the shaft 86 and the adjustable cutter 82, the adjustable cutter 82 can be positioned at any desired location and/or orientation as needed to cut, measure, position, and/or remove tissue. The adjustable cutter 82 may include sharp edges on one side to facilitate cutting.

FIG. 8 illustrates a side view of the custom device 70 with the adjustable cutter 82 initially co-linear with the shaft 86 (FIG. 8A), as well as with the adjustable cutter 82 rotated about the movable joint 84, thereby forming an angle with the shaft 86 (FIG. 8B). In some embodiments, at least one cable (i.e., the cable used for adjusting the angle of the adjustable cutter) may also facilitate adjustment of the size of an adjustable cutter 82.

FIG. 8C illustrates an enlarged view of the custom device 70, according to aspects of the present embodiments. The custom device 70 may include a connection point 83 connected to the distal end of the adjustable cutter 82 to allow a cable 94 to be coupled thereto. The custom device 70 may also include a transition piece 85 disposed between the adjustable cutter 82 and the moveable joint, which may include one or more pins 89 for rotating the adjustable cutter 82. The custom device 70 may also include a coupling piece 87 that mates with and couples to the shaft 86 such that a distal assembly 91 may be formed or fabricated separate from the shaft 86, and then subsequently coupled thereto. In some embodiments, the distal assembly 91 may be interchangeable such that other tools and or work pieces may be swapped out with the adjustable cutter 82, as needed.

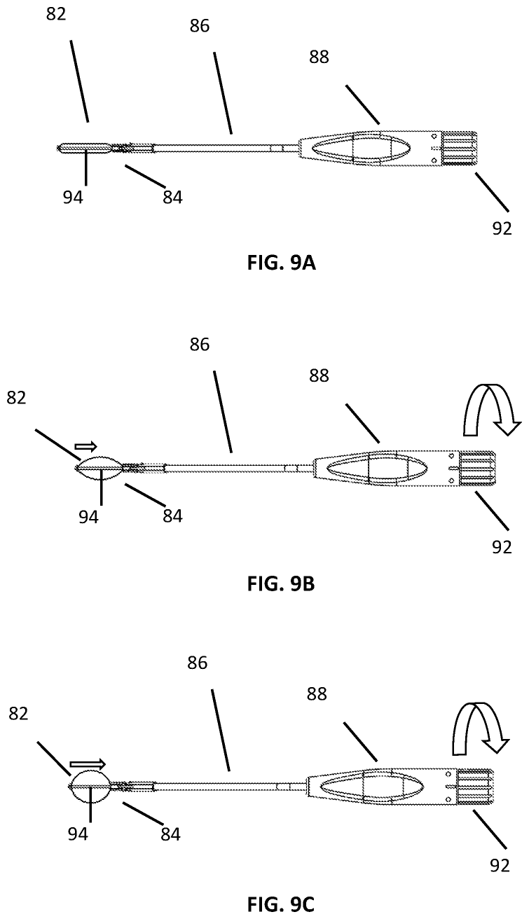

FIG. 9 illustrates multiple views of a custom device 70 comprising at least one cable that may facilitate adjustment of the size of an adjustable cutter 82, according to aspects of the present embodiments. In some embodiments, a distal end of a cable 94 is attached to the interior face of a distal side of an adjustable cutter 82. In some embodiments, an adjusting knob 92 is coupled to a proximal end of a cable that, upon rotation of the adjusting knob 92, exerts a force resulting in a change in a shape of a cutter 82, as shown in FIG. 9.

In some embodiments, an adjusting knob 92 is coupled to a single cable that, upon rotation of the adjusting knob 92, exerts a force resulting in a change in both an angle of a joint 84 and a shape of a cutter 82. For example, the adjusting knob 92 may be initially rotated such that the cable 94 rotates adjustable cutter 82 about the movable joint 84 until the adjustable cutter 82 reaches a predetermined and/or desired angle (for example, 45 degrees, or from about zero degrees to about 180 degrees, as described herein). Continued rotation of the adjustable knob 92 (i.e., beyond the point where the adjustable cutter 82 has reached the predetermined angle) may then cause the adjustable cutter 82 to begin to flex, as shown in FIGS. 9B and 9C. Whereas the adjustable cutter 82 appears substantially linear in FIG. 9A (allowing for some thickness and contouring at the distal end), the adjustable cutter 82 becomes more oval-shaped in FIG. 9B, and more circular-shaped in FIG. 9C.

In some embodiments, an adjusting knob is coupled to at least two cables (e.g., at least a first cable and a second cable). In some embodiments, an adjusting knob 92 is coupled to (1) a first cable that, upon rotation of the adjusting knob 92, exerts a force resulting in a change in both an angle of a joint 84, and (2) a second cable that upon further rotation of the adjusting knob 92, exerts a force resulting in a shape of a cutter 82. The custom device 70 may also include a single cable 94 that is looped through a portion of the adjustable cutter 82 to help facilitate both the changing of the angle of the adjustable cutter 82, as well as the changing of the shape of the adjustable cutter 82.

Referring still to FIG. 9, the custom device 70 may be returned to its original shape and orientation via one or more mechanisms, as described herein. In one embodiment, the inherent elasticity of the adjustable cutter 82 may be biased to the more linear shape shown in FIG. 9A, such that as the adjustable knob 92 is rotated in the opposite direction, the adjustable cutter 82 is restored to its original shape, pulling the one or more cables 94 with it. Similarly, the moveable joint 84 may include one or more springs (for example, a hinged spring) biased to the linear or zero-degree orientation of the adjustable cutter 82 relative to the shaft 86 such that as the adjustable knob 92 continues to be rotated in the opposite direction past the point at which the shape of the adjustable cutter 82 has returned to its original linear shape (shown in FIG. 9A), the spring in the movable joint 84 pushes or pulls the adjustable cutter 82 back to zero degrees relative to the shaft 86. In some embodiments, a second cable may be used in addition to, or in place of, the inherent elasticity of the adjustable cutter 82 and/or hinged spring in the movable joint 84, in order to restore the custom device back to its original shape and orientation. In some embodiments, one or more linkages may be used in addition to, or in place of, the one or more cables. In some embodiments, an adjustable cutting device comprising an adjustable cutter 82 may be used to measure or approximate the size and/or shape of a defect in a surgical site. In some embodiments, a proximal end of a handle 88 comprises indicators of the size and/or position of an adjustable cutter 82 and/or joint 84 relative to a position of an adjustable knob 92. In some embodiments, the cable 94 may be a solid linkage 94. For example, in embodiments that include one or more solid linkages 94 rather than cables 94, the user may exert both a pulling and pushing force on the one or more solid linkages via the knob 92. In such embodiments that include one or more solid linkages 94, the inherent elasticity of the adjustable cutter 82 may not be needed to restore the position and shape of the adjustable cutter 82 back to its original (fully extended) shape, since the one or more solid linkages 94 may be used to push the adjustable cutter 82 back to its original shape.

In some embodiments, an adjustable cutting device may be used to perform methods described herein.

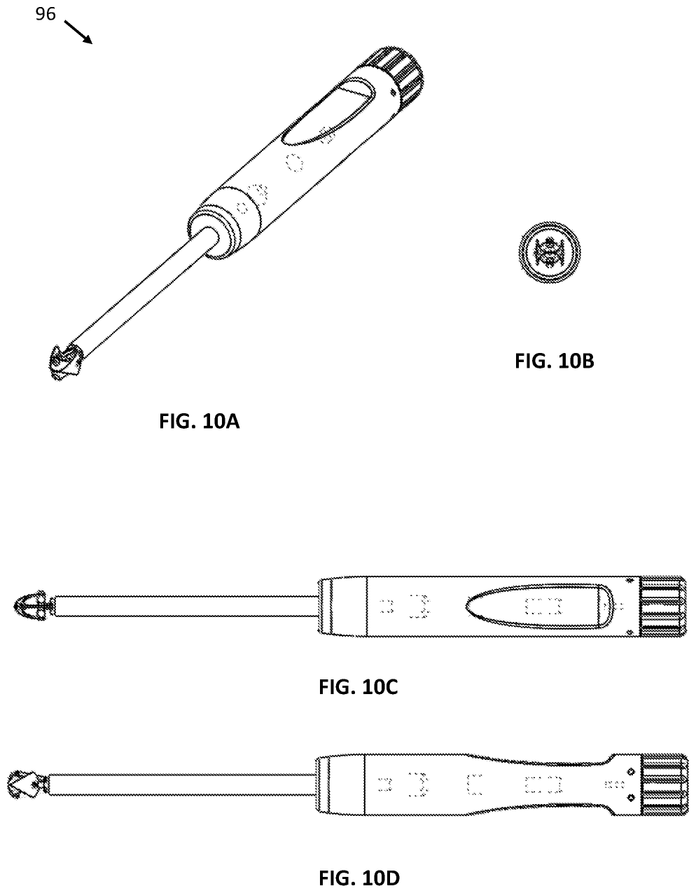

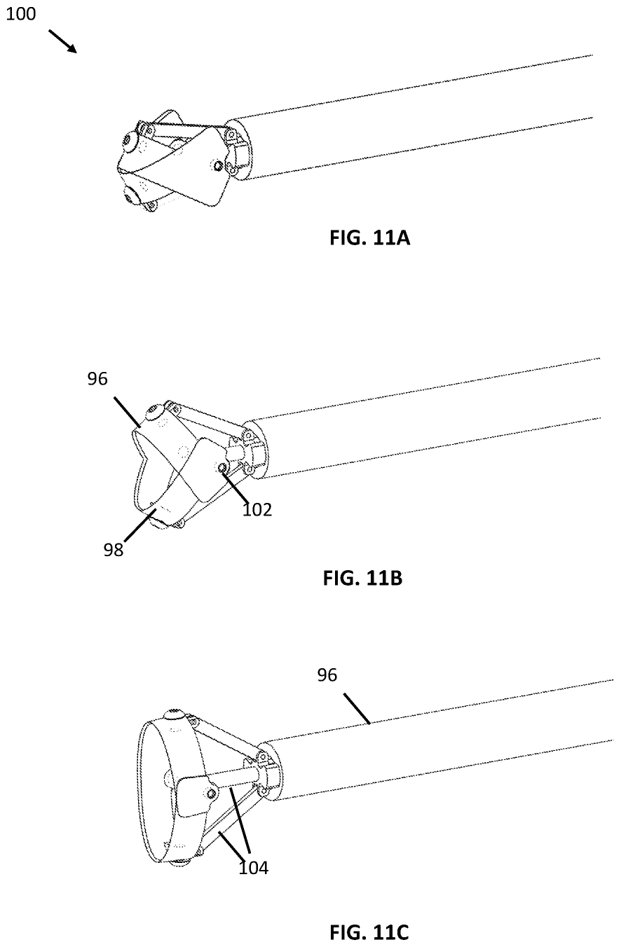

FIGS. 10 and 11 illustrate an alternate embodiment of a custom device 100, according to aspects of the present embodiments. FIG. 10 shows front, side, top, and perspective views of the custom device 100. FIG. 11 illustrates an enlarged view of the distal end of the custom device 100, which includes first and second cutting teeth 96, 98. The first and second cutting teeth 96, 98 may be hinged together via one or more pins 102. The cable or cables 94 may be connected to the one or more pins 102, thereby allowing the first and second cutting teeth 96, 98 to expand open and/or rotate closed. For example, in FIG. 11A, the first and second cutting teeth 96, 98 are in a closed position, allowing the custom device 100 to fit through the cannula 54. In some embodiments, the cable 94 may be a solid linkage 94. For example, in embodiments that include one or more solid linkages 94 rather than cables 94, the user may exert both a pulling and pushing force on the one or more solid linkages via the knob 92. In such embodiments that include one or more solid linkages 94, the inherent elasticity of the adjustable cutter 82 may not be needed to restore the position and shape of the adjustable cutter 82 back to its original (fully extended) shape, since the one or more solid linkages 94 may be used to push the adjustable cutter 82 back to its original shape. Once the first and second cutting teeth 96, 98 are through the distal end of the cannula 54, the first and second cutting teeth 96, 98 may partially expand open (FIG. 11B) or may fully expand (FIG. 11C) by releasing the cable. The custom device 100 may include a hinged spring such that it naturally opens to the fully open position of FIG. 11C, when the cable is not pulled. As such, in order to retract the first and second cutting teeth 96, 98, the cable may be pulled, which pulls the pin 102 closer to the distal end of the shaft and rotates the first and second cutting teeth 96, 98 to the closed or retracted position as shown in FIG. 11A. A plurality of linkages (for example, 2, 3, 4, and/or more than 4) may be used to couple the first and second cutting teeth 96, 98 to the distal end of the shaft 96. Each of first and second cutting teeth 96, 98 may include a geometry that is substantially semi-circular and/or curvilinear, as shown in FIG. 11. Although not shown, the embodiments of the custom device 100 depicted in FIGS. 10 and 11 may include a knob 92 that functions similar to the knob 92 depicted in FIGS. 7-9.

The custom device 100 shown in FIGS. 10 and 11 may be used to measure, cut, extract, and/or position tissue similar to how the custom device 70 of FIGS. 7-9 may be used. In some embodiments, the custom devices 70, 100 of FIGS. 7-11 may include a proximal slider (that moves forward and backwards parallel to a central axis of the handle 88) to adjust the adjustable distal tip 82 angle (i.e., the angle between the adjustable cutter 82 and the shaft 86) and/or the rotation of the first and second cutting teeth 96, 98 instead of the adjustable knob 92. In some embodiments, the proximal slider may also be used to adjust the shape of the adjustable cutter 82.

In some embodiments, a kit of the invention may provide sterile components suitable for easy use in the surgical environment, and/or may provide a suitable hemostatic barrier, suitable covering patch, and/or, organic glue.

In some embodiments, a kit may include sterile, cell-free matrix material suitable for supporting autologous chondrocytes or allogeneic chondrocytes, for example that may be suitable for implanting into an articular joint surface defect.

In some particular embodiments, a suitable hemostatic barrier (e.g., that may be included in a kit and/or be otherwise utilized in accordance with the present invention) may be or include, for example, a Surgicel® hemostatic barrier.

In some particular embodiments, a suitable covering patch (e.g., that may be included in a kit and/or otherwise utilized in accordance with the present invention) may be or include a Bio-Gide® covering patch.

In some embodiments, a hemostatic barrier (e.g., a Surgicel®) hemostatic barrier) and/or a covering patch (e.g., an ACI-Maix® covering patch) may include a glue, e.g., a tissue glue, which, in some embodiments, may be an organic glue (e.g., a Tisseel® organic glue). In some embodiments, glue may be applied (e.g., as a covering) so that time to resorption is increased (e.g., as exemplified herein, for example, in Example 8).

In some embodiments, a hemostatic barrier (e.g., a Surgicel® hemostatic barrier) and/or a covering patch (e.g., a Bio-Gide® covering patch), and in particular one treated with a glue (e.g., may include a Tisseel® organic glue) may be supplemented with aprotinin (e.g., in a manner and/or to an extent that time to resorption is increased).

In some embodiments, a hemostatic barrier and covering-patch may be both a semi-permeable collagen matrix which is treated to extend the time until resorption of the material. It is also possible to provide Tisseel® glue in enhanced form as a separate component to be applied as needed because of the inherent variability and unique circumstances every repair/transplantation procedure will encounter.

In some embodiments, a kit may include a surgical instrument or multiple surgical instruments. In some embodiments, a kit may include one or more cannulas (e.g., 1, 2, 3, 4, 5, or 10 or more cannulas). In some embodiments, a kit may include a cannula or multiple cannulas having inner diameters within a range from about 5 mm to about 15 mm, about 6 mm to about 12 mm, about 7 mm to about 11 mm, about 8 to about 9 mm, or about 8.5 mm. In some embodiments, cannulas may be composed of a material selected from the group consisting of plastics, metals, rubber, silicone, fiber glass, and combinations thereof (for example, composite materials). In some embodiments, a kit may include 2 cannulas, 1 of which is used for the loading of a composition (a loading cannula), and 1 of which is positioned in a surgical site for the delivery of a composition to tissue in the surgical site (a delivering cannula). In some further embodiments, a kit may include an adapter for use in connecting a loading cannula and a delivering cannula in order to facilitate the delivery of the composition from the distal end of the lading cannula to tissue in the surgical site. In some embodiments, cannulas may have lengths specifically suited to the treatment of a defect in a specified joint.

In some embodiments, a kit may include cells seeded on a surface of a matrix. In some embodiments, cells may include allogeneic chondrocyte cells. In some embodiments, cells may include cells obtained from a non-human source.

In some embodiments, a kit may include tools for pulling (i.e. pullers) a composition into a cannula (which cannula may optionally also be included in the kit).

In some embodiments, a puller may be a surgical grasper. In some embodiments, a surgical grasper may be an arthroscopic grasper. In some embodiments, a surgical grasper may be a small joint grasper. In some embodiments, a surgical grasper may comprise atraumatic jaws. In some embodiments, a surgical grasper can be inserted into a cannula (which cannula may optionally also be included in the kit), such that its shaft is disposed within the lumen such that the jaws protrude from the distal end of the cannula. In some embodiments, pullers may have lengths specifically suited to the treatment of a defect in a specified joint.

In some embodiments, a kit may include a templating backing material. In some embodiments, a templating backing material may include a bandage, such as an Esmarch bandage. Other templating backing materials may include sterile aluminum foil, paper, and/or other materials. In some embodiments, a kit may include a sterile ink marker. In some embodiments, a kit may include a sterile ruler.

In some embodiments, a kit may include one or more tools for cutting and shaping a templating backing material or composition that may be used for treatment. In some embodiments, a kit may include scissors, razor blades, scalpels, custom cutters (surgical cookie cutters), cutting blocks, surgical mallets, ring curettes, and/or cutting needles. In some embodiments, custom cutters provided may be round. In some embodiments, custom cutters may be oval-shaped. In some embodiments, custom cutters may be oblong-shaped.

In some embodiments, a kit may include at least one custom device. In some embodiments, a kit may include a device that is or comprises an adjustable cutting device. In some embodiments, a kit may include an adjustable cutting device described herein. In some embodiments, a kit may include a device that is or comprises a custom cannula. In some embodiments, a custom cannula provided in a kit may have an inner diameter in a range from about 15 mm to about 20 mm.

In some embodiments, a kit may include one or more tools for securing an implanted composition in a surgical site. In some embodiments, tools for securing an implanted composition may include one or more probes. In some embodiments tools for securing an implanted composition may include an elevator, such as a freer elevator.

In some embodiments, a kit may include forceps. In some embodiments, a kit may include Adson forceps. In some embodiments, a kit may include toothless Adson forceps.

In some embodiments, a kit may include at least one custom surgical grasper and at least one custom cannula comprising a locking mechanism capable of temporarily immobilizing a surgical grasper disposed in the lumen of the cannula.

In some embodiments, a kit may include neurosurgical patties.

In some embodiments, a kit may include sutures.

In some embodiments, a kit may include sterile dishes.

In some embodiments, a kit may include sterile flasks.

In some embodiments, a kit may include sterile solutions. In some embodiments, a kit may include epinephrine. In some embodiments, a kit may include sterile saline.

In embodiments of the kit in which the custom devices illustrated in FIGS. 7-11 are available, a cell-seeded matrix may be positioned according to the description of FIGS. 7-11 above. In embodiments of the kit in which the the custom devices illustrated in FIGS. 7-11 are not available, a cell-seeded matrix may be positioned according to the description of FIGS. 1-5 above. In addition, the custom devices illustrated in FIGS. 7-11 may be used in connection with the methodologies described above relating to FIGS. 1-5.

It will be appreciated by persons skilled in the art that numerous variations and/or modifications may be made to the invention shown in the specific embodiments without departing form the spirit and scope of the invention as broadly described.

All publications, patent applications, patents and other references mentioned herein are incorporated by reference in their entirety. In addition, the materials, methods and examples are illustrative only and not intended to be limiting. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

### EXAMPLES

### Example 1: Defect Preparation

The present example (Example 1) describes the preparation of a knee cartilage defect in surgical site to be treated using the technologies provided in the present disclosure. First, a surgical site may be flushed and washed with isotonic saline. A cartilage defect and the cartilage surrounding the defect may be assessed physically and visually via an arthroscopic device (e.g., arthroscopic camera) inserted into a first surgical site adjacent to the defect via a cannula positioned in the site. Attention should be paid to discoloration, irregular surface areas, absence of normal resiliency, cartilage thinning, and/or unstable and undermined cartilage. After inspection, the area of the defect may be outlined and sculpted using, for example, without limitation, a custom cutter or scalpel that is inserted into a cannula in a second surgical site. The defect should be debrided down to the subchondral bone and peripherally until vertical walls of healthy, stable cartilage surrounds the defect site. All damaged and fibrous tissue on the defect bed should be removed. Care should be taken such that removal of healthy cartilage is minimal. Care should also be taken to avoid penetrating the subchondral bone. A knee joint may be drained of fluid through an incision or via suction, in preparation for the delivery of an implant to a defect in a surgical site. Excess fluid around the defect can also be dried using kittner dissectors (“peanut” sponges), in effect wicking excess fluid away from the cartilage defect. For punctate bleeding from the subchondral bone, hemostasis may be achieved by pressure with diluted epinephrine-soaked neurosurgical patties (1 cc of 1:1000 Epinephrine diluted with 20 cc of sterile saline) or by applying fibrin sealant at the point of bleeding.

### Example 2: Templating

The present example (Example 2) describes a templating step that may be performed prior to MACI implantation. The goal of the templating step is to approximate the size and shape of the defect before the MACI implant is unpacked and prepared for delivery, thus minimizing the time from implant preparation to delivery, and thus preserving cell viability and increasing the likelihood of a successful treatment. The templating step should be performed after defect preparation (described in Example 1). A template of the detect may be created by (1) passing a templating material (e.g., paper, sterile aluminum foil, etc.) through the cannula and into the surgical site, adjacent to the defect. (2) removing the templating material and trimming it to more closely approximate the size and shape of the defect, (3) reinserting the templating material into the surgical site via the cannula to again approximate the size and shape of the defect, and (4) removing the templating material from the surgical site and repeating steps 1-3 until a template that closely matches the size and shape of the defect is achieved. Throughout the process, a sterile marking pen may be used to mark the template in order to help keep track of the orientation of the template with respect to the defect. Care should be taken to ensure the proper orientation of the template with respect to rotation and with the correct surface facing into the defect. In some embodiments, a cell-seeded matrix may be placed on top of the templating material, cell-side up. The side of the template facing into the defect will be the side onto which a cell-seeded matrix may be placed, cell-side up, so that the cells do not contact the templating material. The cell-seeded matrix may then be shaped to match the size and shape of the templating material. In some embodiments, the cell-seeded matrix may be delivered together with the templating material in the orientation described above. In such a case, the templating material may provide additional structural support for the cell-seeded matrix during delivery.

### Example 3: Delivery by Unfolded Method

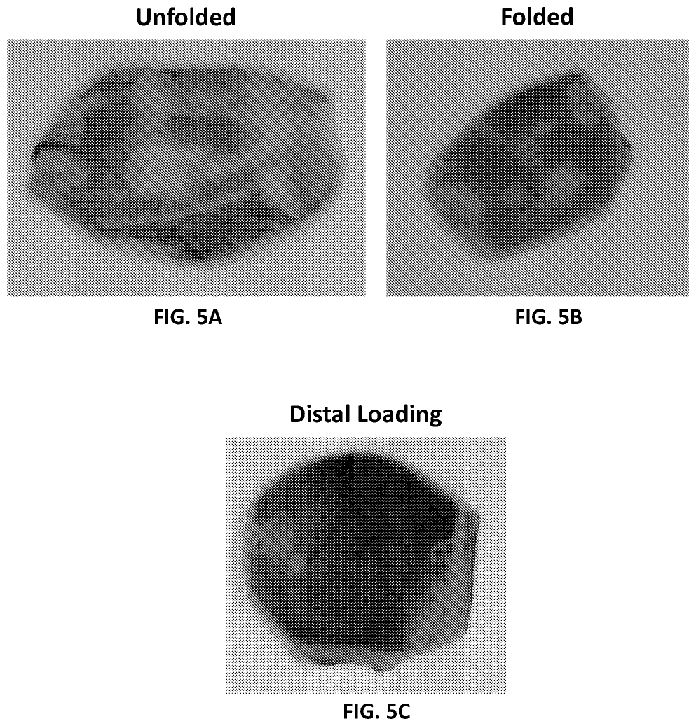

The present Example (Example 3) describes an arthroscopic method for the delivery of a cell-seeded matrix to a defect in a site in an articulating joint. First, the user may grasp one edge of a cell-seeded matrix using a surgical grasper in a manner that minimizes contact with cells on the matrix (See FIG. 2A), as grasping the matrix with the surgical grasper results in loss of cells seeded in the grasped location (see FIG. 5). Next, the user may insert the jaws of the surgical grasper into a cannula such that the grasped edge of the cell-seeded matrix is the first part of the matrix to enter the cannula (see FIG. 2B-C). The user, while still grasping the matrix with the surgical grasper, may then proceed to push the matrix through the entire length of the cannula until the jaws and the matrix exit the distal end of the cannula (see FIG. 2D). If the cannula is positioned in a surgical site, the matrix may then be delivered to a defect in tissue in the site, with the cell-seeded side of the matrix contacting the tissue in the site. The matrix may then be secured in the site by applying gentle force with a knee probe or freer elevator, followed by fibrin glue fixation (see Example 8 and FIG. 1D).

### Example 4: Delivery by Folded Method

The present Example (Example 4) describes an arthroscopic method for the delivery of a cell-seeded matrix to a defect in a site in an articulating joint that offers an alternative to the method described in Example 3. First, the user folds a cell-seeded matrix in half—without creasing the matrix—such that the matrix forms a shape reminiscent of a folded circle (i.e., a circle folded around a central axis that is parallel to its pre-folded plane). Stated otherwise, the cell-seeded matrix may be folded in a manner such that it resembles the shape of a taco. The matrix can be folded such that the cell-seeded surface is on the inside surface or the outside surface of the folded matrix. Next, the user grasps the folded matrix where the two edges meet using a surgical grasper (see FIG. 3A). The user then inserts the jaws of the surgical grasper into a cannula such that the grasped portion of the cell-seeded matrix is the first part of the matrix to enter the cannula (see FIG. 3B-C). The user, while still grasping the matrix with the surgical grasper, then proceeds to push the matrix through the entire length of the cannula until the jaws and the matrix exit the distal end of the cannula (se FIG. 3D). If the cannula is positioned in a surgical site, the matrix can then be delivered to a defect in tissue in the site, with the cell-seeded side of the matrix contacting the tissue in the site. The matrix can then be secured in the site by applying gentle force with a knee probe or freer elevator, followed by fibrin glue fixation (See Example 8 and FIG. 1D).

The orientation of the cells on the folded matrix presents a number of benefits and draw backs that must be assessed by the user prior to loading the matrix into the cannula. If the cell-seeded side of the matrix is on the outside of the folded matrix, the cells will be in the preferred orientation (i.e., cells facing the defect) upon entry to the surgical site, which will reduce the time and physical manipulation necessary for the user to contact the defect in the site cell-side down. However, some cells may be lost and/or damaged as the matrix traverses the cannula, which may negatively impact the health of the implanted cells, and thus may decrease the likelihood of a successful treatment outcome. A shorter cannula may be preferred when delivering a matrix to a site using this method and cell orientation. If the cell-seeded side of the matrix is on the inside of the folded matrix, the cells will be in the non-preferred orientation (i.e., cells facing away from the defect) upon entry to the surgical site, which will increase the time and physical manipulation necessary for the user to contact the defect in the site cell-side down. While the increased time and physical manipulation may carry negative consequences for cell health and viability, the delivered cells may be much less likely to be damaged by contacting the interior wall of the cannula.

### Example 5: Delivery by Distal Loading Method

The present Example (Example 5) describes an arthroscopic method that may achieve certain surprising improvements relative, for example, to delivery methods described in Example 3 and Example 4. Among other things, the technology described in the present Example embodies an insight relating to identification of the source of a problem with certain alternative arthroscopic delivery strategies in which cell viability is decreased, potentially due to contact between cells on a support matrix and the interior wall of a cannula used to deliver the support matrix-seeded cells to their target in a surgical site (e.g., as described in Example 3 and Example 4).

In the present Example, an arthroscopic grasper is disposed in the lumen of a cannula prior to any contact with a composition of the invention, including a cell-seeded support matrix that has a cell-seeded surface and a non-cell-seeded surface. The arthroscopic grasper is disposed such that the jaws protrude from the distal end of the cannula. The user grasps the cell-seeded support matrix at the edge, taking care to minimize contact between the jaws and the center of the matrix. The portion of the matrix that is grasped using the jaws of the grasper has been shown to be devoid of viable cells (See FIG. 5C). The user then pulls the cell-seeded support matrix into the cannula such that the non-cell-seeded side of the support matrix contacts the interior wall of the cannula, and the cell-seeded surface faces the cannula lumen (see FIG. 4B-C). This may represent an improvement over the methods described in Example 3 and Example 4 above because in such Examples, the implant must traverse the entire length of the cannula before deposition into the surgical site. In the present Example (Example 5), the implant must only traverse a distance that is about equal to the length of the implant along an axis parallel to the cannula. An additional improvement in the present Example is that the cell-seeded surface of the matrix is inherently less likely to contact the cannula interior wall than a matrix delivered using the method of Example 3, and the method of Example 4 in which the cell-seeded surface is the outside surface of the folded matrix.

### Example 6: Evaluation of Cell Number and Viability Across Various Delivery Methods Using a Human Cadaver Knee Model

In the present Example (Example 6), the delivery methods described in Examples 3, 4, and 5 are compared on the basis of their impact to cell number and viability following simulated delivery to a defect in a surgical site in knee joint tissue. The present experiments were performed using a human cadaver knee model. Positive controls include a condition in which a cell-seeded matrix was not delivered to a site by any method, as well as a condition in which a cell-seeded matrix was delivered to a site using a traditional open surgical technique.

When compared to cell-seeded matrices delivered arthroscopically using the “unfolded” and “folded” methods, matrices delivered using the distal loading technique exhibited a greater average cell number per implant as determined by a minimum cell number assay. Briefly, 6-mm punches from each matrix were removed following simulated delivery to a defect in a cadaver knee. Each punch was placed in one well of a 96-well plate with matrix transport media and allowed to incubate for 1 to 3 hours at 37° C. After incubation, half of conditioned media (containing protease released from dead cells) was placed in a separate well. Bis-AAF-R110 protease substrate (from Promega MultiTox-Fluor Multiplex Cytotoxicity assay) was mixed with saponin and phenol red in assay buffer to make a “mastermix”. The final concentration of various components in the mastermix was: 0.83 mM bis-AAF-R110 substrate, 1.67% saponin, 0.167 mg/mL phenol red. The mastermix was added to the wells containing matrix with conditioned media and conditioned media alone. After a 60-minute incubation, the signal generated by the protease was read using a fluorescent plate reader at excitation 485 nm and emission 520 nm from both the wells containing matrix with conditioned media and conditioned media alone. The minimum cell number was measured in Relative Fluorescence Units (RFUs), which is the fluorescent signal generated by proteolytic cleavage of the bis-AAF-R110 substrate. The RFU measurement is calculated from the normalized signal from the live cells excluding the dead cells. The final RFU value for minimum cell detection is calculated using the following formula:

$$Normalized Signal from Live Cells = ( Normalized Signal from Punches ) - ( Normalized Signal from Conditioned Media )$$

An RFU signal of at least 8500 indicates that the product contains a minimum of 5×10⁵ cells/cm².

Delivery via the distal loading method yielded the unexpected and surprising result that the average cell number was similar to that of positive control samples that did not undergo delivery to a site (see FIG. 6A). As a qualitative visual measure of cell viability, cell metabolic activity was determined by staining cells on a matrix with 3-(4,5-dimethylthiazol-2-yl)-2,5-diphenyltetrazolium bromide (MTT), and qualitatively examining cells positive for insoluble formazan, a dark-colored conversion product that marks actively respiring cells. Compared to the other methods tested, matrices delivered using the distal loading method retained the highest number of metabolically active cells, approaching that of undelivered positive control matrices (see FIG. 6B). Performing the distal loading method on a matrix twice (to simulate circumstances under which a delivered implant must be removed to correct the implant size and/or shape prior to implantation) predictably resulted in a decrease in cell number (see FIG. 6A) and cell viability (see FIG. 6B), but not below acceptable threshold levels. Surprisingly, the mean cell number and percent cell viability observed on matrices delivered via the distal loading method surpassed those observed on matrices delivered with the traditional open surgical technique with thumb pressure.

### Example 7: Delivery by Distal Loading Method-Multiple Cannulas

The present Example (Example 7) describes an arthroscopic method that is an extension of that described in Example 5. In Example 7, a cell-seeded matrix can be delivered to a defect in a surgical site using more than one cannula. The user may load a cell-seeded matrix into the distal end of a first cannula (loading cannula) according to the methods described in Example 5. A second cannula (delivering cannula) may be positioned in a surgical site adjacent to a defect to be treated. The distal end of the loading cannula containing the loaded cell-seeded matrix can be connected to the protruding end of the delivering cannula-potentially through the use of an adaptor component-such that the matrix can be pushed into and through the delivering cannula and into the surgical site. One potential advantage to this technique over the technique described in Example 5 is that the technique described in Example 5 requires additional time to position the single cannula in the site, which may have negative consequences for cell health and viability. Importantly, it should be noted however that in the method described in the present Example (Example 7), the matrix must traverse the entire length of the delivering cannula, increasingly the likelihood of applying unintended forces to the cells on the matrix that may negatively impact cell health. Accordingly, the method described in the present Example is optimally performed using a cannula with a length that is as short as is feasible (e.g., about 2 cm to about 5 cm) given the conditions of the surgery.

### Example 8: Securing a MACI Implant in a Knee Cartilage Defect

The present example (Example 8) describes a method for securing a MACI implant in a defect in a surgical site. An implant may be secured using a fibrin glue fixation step that may be performed following arthroscopic delivery of a MACI implant to a defect in a patient. After the implant is inserted into the defect, with the cell-seeded side of the implant facing the defect, fibrin sealant (such as Tisseel®, fibrin based adhesive available from Baxter, Austria) may be applied to the rim (i.e., periphery) of the implant. Light pressure may then be applied using a surgical tool, such as a surgical probe, a Howarth or Freer Elevator, or another tool. Interrupted sutures using 6.0 Vicryl® may be used to secure the implant if desired or if conditions warrant, particularly if the defect is uncontained or the lesion is larger than 10 cm².

The security of the implant should be tested by fully flexing and extending the knee several times, and then inspecting the implant to ensure that it has remained in place. The joint may than be irrigated in order to remove any remaining free particles of bone or cartilage in the site. Care should be taken to ensure that the implant is protected and not dislodged during irrigation. The wound may then be closed using standard techniques known to those skilled in the art.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. An arthroscopic surgical method comprising:
making at least two incisions in a subject adjacent to a defect to be treated arthroscopically, the at least two incisions comprising a first incision and a second incision;
inserting a first cannula into the first incision;
preparing a site of the defect to be treated at a joint in the subject;
shaping a template material to match a shape of the prepared defect site to form a shaped template material;
shaping a cell-seeded matrix to match a shape of the shaped template material by placing the cell-seeded matrix on top of the template material with the cells facing up and cutting the cell-seeded matrix to match the shape of the shaped template material;
delivering the cell-seeded matrix to the prepared defect site through the first cannula; and
fixating the delivered cell-seeded matrix using a glue.

2. The method of claim 1, wherein the defect comprises a cartilage defect, and wherein the joint is one of a knee, an ankle, an elbow, a shoulder, a hip, or a wrist of the subject.

3. The method of claim 1, wherein preparing the defect site comprises:
flushing the defect site;
assessing and/or measuring the defect site;
outlining the defect site;
sculpting the defect site to remove damaged tissue; and
debriding the cartilage down to subchondral bone.

4. The method of claim 1, wherein the template material comprises at least one member of the group consisting of sterile aluminum foil, sterile paper, and an Esmarch bandage.

5. The method of claim 1, wherein shaping the template material comprises:
(a) passing the template material through the first cannula inserted at the first incision;
(b) observing the template material adjacent to the defect site;
(c) removing the template material from the first cannula and cutting it to approximate the size and/or shape of the defect site based on the observations; and
(d) repeating steps (a)-(c) until the template material matches the size and/or shape of the defect site.

6. The method of claim 1, wherein the cell-seeded matrix comprises at least one of a bioresorbable material and collagen to form a matrix, and
wherein the cells are seeded on a surface of the matrix at a concentration of at least 250,000 cells/cm².

7. The method of claim 1, wherein the cells comprise chondrocytes.

8. The method of claim 1, wherein the cells comprise at least one of cells autologous to the subject and allogeneic cells.

9. The method of claim 1, wherein delivering the cell-seeded matrix comprises:
grasping the cell-seeded matrix using a surgical grasper;
bringing the unseeded surface of the cell-seeded matrix into contact with a proximal opening of the first cannula inserted into the first incision;
pushing the cell-seeded matrix into a lumen of the cannula; and
using the surgical grasper to push the cell-seeded matrix through to a distal end of the cannula.

10. The method of claim 1, wherein delivering the cell-seeded matrix comprises:
folding the cell-seeded matrix in half;
grasping the folded cell-seeded matrix using a surgical grasper;
bringing the folded cell-seeded matrix into contact with a proximal opening of the first cannula inserted at the first incision;
pushing the folded cell-seeded matrix into a lumen of the first cannula inserted at the first incision; and
using the surgical grasper to push the folded cell-seeded matrix through to a distal end of the cannula.

11. The method of claim 1, wherein delivering the cell-seeded matrix comprises:
prior to inserting the first cannula into the first incision, disposing a surgical tool in a lumen of the first cannula such that jaws of the surgical tool protrude from a distal end of the first cannula and a handle of the surgical tool protrude from a proximal opening of the first cannula;
grasping the cell-seeded matrix using the jaws of the surgical tool;
pulling the cell-seeded matrix into the distal end of the first cannula using the surgical tool such that the cell-seeded surface faces a lumen of the first cannula and the cell-seeded matrix is partially folded;
inserting the first cannula into the first incision while the cell-seeded matrix and surgical tool are disposed within the lumen of the first cannula; and
using the surgical tool to push the cell-seeded matrix out of the distal end of the first cannula such that the cell-seeded matrix unfolds.

12. The method of claim 11, wherein inserting the first cannula comprises inserting the first cannula into a third cannula that has previously been inserted into the first incision.

13. The method of claim 1, wherein each cannula comprises an inner diameter in a range from about 5 mm to about 20 mm and a length in a range from about 20 mm to about 240 mm.

14. The method of claim 1, wherein an area of the defect and the area of the cell-seeded matrix after shaping are between about 1 cm² and about 10 cm².

15. A surgical kit comprising:
two or more cannulas;
a cell-seeded matrix comprising a bioresorbable support matrix and a plurality of cells seeded on a surface of the bioresorbable support matrix at a concentration of at least 250,000 cells/cm²;
a surgical grasper;
a templating material;
one or more tools for shaping the template material and the cell-seeded matrix;
one or more tools for outlining, cutting, and debriding cartilage.

16. The kit of claim 15, wherein the two or more cannulas each comprise an inner diameter in a range from about 5 mm to about 20 mm and a length in a range from about 20 mm to about 240 mm.

17. The kit of claim 15, wherein the surgical grasper comprises a shaft and jaws, wherein the jaws comprise atraumatic jaws.

18. The kit of claim 15, wherein the one or more tools for shaping comprise members selected from the group consisting of scissors, razor blades, scalpels, custom cutters, cutting blocks, surgical mallets, ring curettes, tweezers, and cutting needles.

19. The kit of claim 15, wherein the one or more tools for shaping comprise custom cutters with blades shaped as a circle or an oval.

20. A custom surgical device comprising:
a handle;
an adjustable knob rotatably coupled to a proximal end of the handle;
a shaft coupled to the distal end of the handle;
a movable joint coupled to the distal end of the shaft; and
an adjustable distal end coupled to the moveable joint,
wherein rotating the adjustable knob causes the adjustable distal end to rotate about the moveable joint such that an angle between the adjustable distal end and the shaft changes.

21.-24. (canceled)
